(19) United States
(12) Patent Application Publication
(10) Pub. No.: US 2012/0157339 A1
(43) Pub. Date: Jun. 21, 2012

(54) Molecular Markers and Assay Methods for Characterizing Cells

(76) Inventors: Guoping Fan, Agoura Hills, CA (US); Anyou Wang, Los Angeles, CA (US)

(21) Appl. No.: 13/379,543

(22) PCT Filed: Jun. 29, 2010
(86) PCT No.: PCT/US10/40388

§ 371 Date: Dec. 20, 2011

Related U.S. Application Data
(60) Provisional application No. 61/221,208, filed on Jun. 29, 2009.

(51) Int. Cl. C40B 30/04; C12Q 1/68

(52) U.S. Cl. 506/9; 435/6.11

(57) ABSTRACT

Disclosed herein are molecular markers and assay methods for characterizing cells. As disclosed, the methods entail determining a methylation state of at least one CpG in a region of a nucleotide molecule of the cell, comparing the methylation state with that of a corresponding CpG of a comparison cell of a known cell type, a known cell line, or a known cell strain, and distinguishing, identifying or designating the cell type, the cell line or the cell strain of the cell based on whether the methylation state is the same or different from that of the corresponding CpG.

### CROSS-REFERENCE TO RELATED APPLICATIONS

This application claims the benefit of U.S. Provisional Application Ser. No. 61/221,208, filed 29 Jun. 2009, which is herein incorporated by reference in its entirety.

### BACKGROUND OF THE INVENTION

1. Field of the Invention

The present invention generally relates to molecular markers for characterizing induced pluripotent stem cells (iPSCs), embryonic stem cells (ESCs), and a variety of somatic cells and methods of using thereof.

2. Description of the Related Art

A CpG site (CpG) refers to a cytosine nucleotide occurring next to a guanine nucleotide in the linear sequence of bases along the length of a nucleic acid molecule. The cytosines in CpGs may be methylated or unmethylated. A CpG may be found in a CpG island which is genomic region containing a high CpG frequency. In mammalian genomes, CpG islands are typically about 300 to 3,000 base pairs in length and are in and near about 40% of gene promoters. Generally, a CpG island is a region with at least 200 bp and with a GC content that is greater than 50% and with an observed/expected CpG ratio that is greater than 0.60. A true CpG island is a genomic region of greater than 500 bp with a GC content greater than 55% and an observed to expected CpG ratio of 0.65.

Changes in CpG methylation has been associated with gene regulation and nuclear reprogramming and many research groups have looked at the overall methylation of CpGs in genes associated with nuclear reprogramming. For example, Dailey et al. identify a cell as being an iPSC based on the overall methylation patterns particular promoters of genes, e.g. OCT4, SOX2, and NANOG, involved in nuclear reprogramming. See e.g. WO 2010/033991.

Deng et al. used padlock probes to examine CpG islands and found that the similarity of overall CpG methylation between human embryonic stem cell (hESC) lines and somatic cells (fibroblasts) was more than that between induced pluripotent stem cell (iPSC) lines and somatic cells (fibroblasts). See Deng et al. (2009) Nature Biotech. 27(4):353-360. In other words, Deng et al. found that hESCs appear to be more similar to fibroblasts than iPSCs are to the fibroblasts.

Doi et al. examined various CpG island shores and indicates that the methylation patterns of such CpGs in somatic cells, iPSCs and hESCs appear to be different. See Doi et al. (2009) Nature Genetics 41(12):1350-1354. As Doi et al. state, however, their study has limitations which include the fact that the array employed does not examine single CpGs and very low density methylation and the iPSCs were derived from a only one cell type.

Unfortunately, these research groups do not examine the differences in methylation of single CpGs and/or provide sufficient information which would enable one to distinguish a given cell line or type from another, e.g. HSF1 vs. HUES7, hNPC-iPSC vs. IMR-90-iPSC, etc., or even reliably determine whether a cell is a somatic cell, an iPSC, an ESC, or a particular variety or type of somatic cell.

### SUMMARY OF THE INVENTION

The present invention relates to methods of characterizing a cell as being of a particular cell type from a predetermined group of cell types comprising cell type A, cell type B, etc., which comprises obtaining cell type methylation profiles for each cell type from known cells of each cell type for a set of CpGs; obtaining a methylation profile of the cell for the set of CpGs; using linear discriminant analysis to obtain a constant values from each one of the cell type methylation profiles; using the constant value to obtain methylation amounts for each cell type; calculating a methylation amount of the cell based on the methylation profile of the cell; determining whether the methylation amount of the cell is similar to one of the methylation amounts determined for the known cell types, and designating the cell accordingly, i.e. characterizing the cell as being of the cell type to which its methylation amount most resembles. In some embodiments, the predetermined group of cell types comprise, consist essentially of or consist of embryonic stem cells, a induced pluripotent stem cells and somatic cells.

In some embodiments, the present invention provides methods of characterizing a cell as being of a particular cell type from a predetermined group of cell types comprising cell type A, cell type B, etc. which comprises obtaining cell type methylation profiles for each cell type from known cells of each cell type for a set of CpGs; obtaining an amount of methylation for each CpG of the set of CpGs for the cell; using linear discriminant analysis to obtain constant values for each of the cell types from the cell type methylation profiles; using linear discriminant analysis to obtain sets of coefficients which correspond to each cell type for the set of CpGs; calculating values using the amounts of methylation by multiplying each of the amounts of methylation with the corresponding coefficients of each set of coefficients to obtain a set of multiplied values, summing the set of multiplied values, and adding the respective constant value; designating the cell as being of the cell type which cell type's constant and coefficients result in the largest value.

In some embodiments, the present invention provides methods of characterizing a cell which comprises determining a methylation state of at least one CpG in a region of a nucleotide molecule of the cell, comparing the methylation state with that of a corresponding CpG of a comparison cell of a known cell type, a known cell line, or a known cell strain, and distinguishing, identifying or designating the cell type, the cell line or the cell strain of the cell based on whether the methylation state is the same or different from that of the corresponding CpG. In some embodiments, the cell is distinguished from the comparison cell where the methylation state is different from that of the corresponding CpG. In some embodiments, the cell type, the cell line or the cell strain of the cell is identified as being the known cell type or the known cell line where the methylation state is the same or substantially similar to that of the corresponding CpG. In some embodiments, the cell type, the cell line or the cell strain of the cell of the cell is designated as being that of the comparison cell where the methylation state is the same or substantially similar to that of the corresponding CpG. In some embodiments, the CpG is in a CpG island, outside of a CpG island, or a promoter. In some embodiments, the methylation states of more than one CpG are determined and compared to the methylation states of the corresponding CpGs of the comparison cell.

In some embodiments, the present invention provides a method of determining whether a first cell is the same or different from a second cell which comprises determining a first methylation profile of the first cell for a plurality of CpGs consisting of at least 11 to 273 CpGs selected from the group consisting of the CpGs of FIG. 6, determining a second methylation profile of the second cell for the plurality of CpGs, comparing the first methylation profile with the second methylation profile, and designating the first cell and the second cell to be the same where the first methylation profile and the second methylation profile are the same or designating the first cell and the second cell to be different where the first methylation profile and the second methylation profile are different.

According to any one of the methods disclosed herein, in some embodiments, the CpG is selected from the group consisting the CpGs of FIG. 6. In some embodiments, the methylation states of at least 11 CpGs, at least 14 CpGs, at least 18 CpGs, at least 39 CpGs, at least 59 CpGs, at least 91 CpGs, at least 94 CpGs, at least 175 CpGs, at least 273 CpGs, and ranges of CpGs therebetween each, e.g. 11 to 14, 11 to 94, 39 to 175, 39 to 273, etc., selected from FIG. 6 may be employed in the methods described herein.

According to any one of the methods disclosed herein, in some embodiments, the cell type is selected from the group consisting human embryonic stem cells (hESCs), a neural precursor cell, human induced pluripotent stem cells, fetal lung fibroblasts, fetal brain tissues, foreskin fibroblasts, fetal lung fibroblasts, differentiated neural precursor cells from hESCs, neural precursor cells differentiated from human iPSCs, retinal pigment epithelial cells from hESCs, and neurons derived from hESCs.

According to any one of the methods disclosed herein, in some embodiments, the cell type or cell line is selected from the group consisting of H1, HSF1, HSF6, HUES7, H9, I6, hNPC.iPS_{—}8, hNPC.iPS_{—}9, hNPC.iPS_{—}1, CCD.1079SK_iPS, IMR.90_iPS, PDB_{—}2lox.5IPS, PDB_{—}2lox.17IPS, PDB_{—}2lox.21IPS, PDB_{—}1lox.17_puro.5IPS, PDB_{—}1lox.17_puro.10IPS, PDB_{—}1lox.21_puro.26IPS, PDB_{—}1lox.21_puro.28IPS, IPS7, IPS14, BJ.IPS, IPS_{—}7_b, IPS14_b, hNPC, CCD.1079SK, IMR.9, XHEF, BJ, and sds2d.

In some embodiments, the present invention provides a method of characterizing a cell as being an induced pluripotent cell or an embryonic stem cell which comprises determining the methylation states of at least 14 CpG selected from Group A consisting of cg11852073, cg26606064, cg06868758, cg08349806, cg00250430, cg05661838, cg20855565, cg09601629, cg12153542, cg20357628, cg23268677, cg15842276, cg15747595, and cg07533148 and/or Group B consisting of cg08763351, cg13798376, cg00461841, cg11328541, cg11799561, cg26059632, cg22940988, cg21021629, cg03165378, cg25539131, cg08818984, cg27388462, cg05950276, cg01804844, cg25423111, cg11378044, cg18118795, cg06509940, cg27649653, cg10977115, cg12775613, cg15536242, cg22324153, cg26866325, cg20484002, cg21784940, cg02245418, cg13086586, cg16168311, cg14223017, cg18509239, cg11908570, cg12368241, cg26815021, cg08028004, cg23504707, cg11456838, cg09212058, cg07703337, cg25023829, cg10742225, cg01796228, cg02845923, cg16152813, cg15171237, cg25943702, cg00011459, cg16546489, cg11631275, cg20275133, cg00131557, cg04835638, cg25156443, cg07105440, cg26360732, cg05306176, cg06310844, cg24562819, cg15864184, cg15781794, cg01081263, cg26014197, cg11368509, cg08651674, cg00376639, cg22722454, cg26717786, cg06499652, cg11698762, cg22085335, cg23943801, cg02631957, cg21260850, cg24471268, cg11654333, cg11594131, and cg02567144, and identifying or designating the cell as being an induced pluripotent cell where the CpG is selected from Group A and is methylated or where the CpG is selected from Group B and is unmethylated, or identifying or designating the cell as being an embryonic stem cell where the CpG is selected from Group A and is unmethylated or where the CpG is selected from Group B and is methylated.

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description serve to explain the principles of the invention.

### DESCRIPTION OF THE DRAWINGS

This invention is further understood by reference to the drawings wherein:

FIG. 1 schematically shows the global gene expression clustering of various somatic cells, iPSCs and hESCs.

FIG. 2 schematically shows the hierarchical clustering of overall gene promoter methylation patterns of various somatic cells, iPSCs and hESCs.

FIG. 3A schematically shows the methylation patterns of CpG islands in the gene body of MSX1 in the indicated cell lines. Black=methylated and white=unmethylated. One circle is a single CpG.

FIG. 3B schematically shows the methylation patterns of CpG islands in the promoter region of ZNF540 in the indicated cell lines. Black=methylated and white=unmethylated. One circle is a single CpG.

FIG. 4 shows an excellent correlation of beta-value with methylation levels as assayed by shotgun bisulfite sequencing (BS-SEQ).

FIG. 5A lists 175 CpGs within 146 genes which show either a significant increase of methylation or decrease of methylation in iPSCs when compared to hESCs and can be used to identify or distinguish iPSCs from either hESCs or parental somatic cells.

FIG. 5B lists 39 CpGs in 31 genes of the 175 CpGs of FIG. 5A which show either a significant increase of methylation or decrease of methylation in iPSCs when compared to hESCs and can be used to identify or distinguish iPSCs from either hESCs or parental somatic cells.

FIG. 5C lists 18 CpGs in 15 genes of the 175 CpGs of FIG. 5A which show either a significant increase of methylation or decrease of methylation in iPSCs when compared to hESCs and can be used to identify or distinguish iPSCs from either hESCs or parental somatic cells.

FIG. 5D lists 11 CpGs of the 175 CpGs of FIG. 5A which show either a significant increase of methylation or decrease of methylation in iPSCs when compared to hESCs and can be used to identify or distinguish iPSCs from either hESCs or parental somatic cells.

FIG. 6 provides a table of all CpGs, as exemplified herein, which are indicated by their respective gene (Gene Name) and the identification number (Illumina ID No.) provided by Illumina Inc. (San Diego, Calif.) along with their associated human chromosome and map location. The beta values, which are an indication of methylation levels for each CpG for the indicated cell lines are shown. A beta value of 0.85 or more indicates the CpG is heavily methylated and a beta value of 0.17 or less indicates little methylation, i.e. beta≧0.85, 0.85>beta≧0.7, 0.7>beta≧0.4, 0.4>beta≧0.17, 0.17>beta≧0.1, or beta<0.1 corresponds to 100%, 75%, 50%, 20%, 5%, and 0% methylation, respectively

FIG. 7 is a list of 273 CpGs, by their Illumina ID No., of which clustering analysis of the beta values of 273 CpGs reveal unique methylation signatures for pluripotent stem cells including both hESCs and iPSCs, that are distinctively different from somatic cells. The statistical analysis of beta values as an indication of methylation levels for each CpG showed that differential methylation between pluripotent stem cells and somatic cells is p<0.05.

FIG. 8 itemizes the CpGs, by their Illumina ID No., which have statistically significant changes in the methylation patterns between the indicated iPSCs and the indicated ESCs.

FIG. 9 itemizes 94 CpGs, by their Illumina ID No., that have statistically significant changes in the methylation patterns between their parental somatic cells and iPSCs or between somatic cells and hESCs.

FIG. 10 lists 91 CpGs, by their Illumina ID No., which were used to characterize the following cell lines: H1, HSF1, HSF6, HUES7, H9, I6, hNPC.iPS_{—}8, hNPC.iPS_{—}9, hNPC.iPS_{—}10, CCD.1079SK_iPS, IMR.90_iPS, PDB_{—}2lox.5IPS, PDB_{—}2lox.17IPS, PDB_{—}2lox.21IPS, PDB_{—}1lox.17_puro.5IPS, PDB_{—}1lox.17_puro.10IPS, PDB_{—}1lox.21_puro.26IPS, PDB_{—}1lox.21_puro.28IPS, IPS7, IPS14, BJ.IPS, IPS_{—}7_b, IPS14_b, hNPC, CCD.1079SK, IMR.90, XHEF, and BJ, fetal connective tissues, and fetal brain tissues. The statistical analysis of beta methylation difference for each CpG was P<0.0055. Clustering analysis of beta values of these 91 CpGs reveal unique methylation signatures for the indicated cell lines.

FIG. 11 lists 59 CpGs, by their Illumina ID No., out of the 91 CpGs of FIG. 10 which can be used to effectively distinguish the indicated hESCs and iPSCs from somatic cells. The statistical analysis of beta methylation difference for each CpG between iPSCs and hESCs was P<0.005. Clustering analysis of beta values reveal unique methylation signatures for the indicated cell lines.

FIG. 12 lists 14 CpGs, by their Illumina ID No., out of the 91 CpGs of FIG. 10 which were used to characterize the following cell lines: H1, HSF1, HSF6, HUES7, H9, hNPC.iPS_{—}8, hNPC.iPS_{—}9, hNPC.iPS_{—}10, CCD.1079SK_iPS, IMR.90_iPS, PDB_{—}2lox.5IPS, PDB_{—}2lox.17IPS, PDB_{—}2lox.21IPS, PDB_{—}1lox.17_puro.5IPS, PDB_{—}1lox.17_puro.10IPS, PDB_{—}1lox.21_puro.26IPS, PDB_{—}1lox.21_puro.28IPS, IPS7, IPS14, BJ.IPS, IPS_{—}7_b, IPS14_b, hNPC, CCD.1079SK, IMR.90, XHEF, and BJ. The statistical analysis of beta methylation difference for each CpG was P<0.002 Clustering analysis of beta values reveal unique methylation signatures for the indicated cell lines.

FIG. 13 graphically shows the distinct distribution and separation of three different classes of cell types (iPSCs, hESCs, and somatic cells (SCs)) based on a statistical formula for a methylation signature composed 14 CpGs as shown in FIG. 12. As shown, the names of the cell lines are indicated in their respective quadrants. The upper left quadrant indicates the cells are hESCs. The lower left quadrant indicates the cells are iPSCs and the lower right quadrant indicates the cells are somatic cells.

FIG. 14 itemizes the cell lines as exemplified herein along with their cell class, cell type and source.

### DETAILED DESCRIPTION OF THE INVENTION

The present invention provides molecular markers—one or more of which may be used to characterize a cell as a somatic cell, an induced pluripotent stem cell (iPSC), an embryonic stem cell (ESC), or a particular cell type, cell line, or cell strain. According to the present invention, each molecular marker is a single CpG which may be methylated or unmethylated. Some of the CpGs may be located in CpG islands and/or located adjacent to gene promoters. In some embodiments, the cells are mammalian cells, preferably human cells. In some embodiments, the present invention provides unique sets of CpGs with different levels of DNA methylation which forms characteristic methylation signatures (i.e. profiles) in various cell types. These unique sets of CpGs may used to characterize a variety of cells.

As used herein, a “cell type” refers to the distinct morphological and/or functional form of a cell. See the World Wide Web at en.wikipedia.org/wiki/List_of_distinct_cell_types_in_the_adult_human_body; and Alberts et al. (2002) MOLECULAR BIOLOGY OF THE CELL, 4^{th} ed., Garland Sciences; and Lanza et al. (2004) Handbook of Stem Cells, Elsevier Academic Press, which are herein incorporated by reference in its entirety. For example, each of human skin fibroblasts, neural precursor cells, embryonic stem cells, induced pluripotent stem cells, and retinal pigment epithelia cells has a unique morphology and cell growth condition. As used herein, a “cell line” refers to a permanently established cell culture that will proliferate indefinitely given appropriate fresh medium and space. A cell line differs from a cell strain in that cell lines have escaped the Hayflick limit and have become immortalized. For example, cell lines include human embryonic stem cells (hESCs), a neural precursor cell, human induced pluripotent stem cells, fetal lung fibroblasts, foreskin fibroblasts, fetal lung fibroblasts, differentiated neural precursor cells from hESCs, neural precursor cells derived from human iPSCs, and retinal pigment epithelial cells from hESCs and iPSCs, as exemplified herein.

As used herein, “characterize” includes “identify” and “designate”. Thus, in some embodiments, the molecular markers may be used to “identify” the identity a given cell as being a particular cell class, e.g. somatic cell, an iPSC, an ESC, or as being a particular cell type and/or cell line with 95-100%, preferably 99-100%, degree of certainty. This is particularly useful for separating morphologically similar cell types such as hESCs and iPSCs. For example, a given cell can be identified as being a somatic cell by a specific methylation signature. As used herein, to “designate” the cell class, cell type or cell line of a given cell means that the given cell is like another cell type or cell line, e.g. the given cell's methylation profile for one or more select molecular markers of interest is the same or substantially similar to that of a known cell type or a known cell line. Designating a given cell as being like another cell does not necessarily mean that the identity of the given cell must be the same as the other cell to which it is compared. In other words, if a given cell is designated as being like an ESC, the given cell does not necessarily have to be an ESC, instead, the given cell could be an iPSC that has a methylation profile which is like an iPSC. Similarly, if a given cell is designated as being like a cell from the IMR-90 cell line (e.g. having a methylation profile similar to a cell from an IMR-90 cell line), the given cell need not actually originate from the IMR-90 cell line, but can be from a different cell line. Likewise, if a given cell is designated as being like a human neuronal precursor cell (e.g. having a methylation profile similar to a human neuronal precursor cell), the given may be a neuronal precursor cell for a species other than human, e.g. a non-human primate or other animal, or the given cell may be a cell type that is different from a neuronal precursor cell, e.g. a glial cell.

### Global Gene Expression Clustering

Microarray experiments, as done by Shen et al. (2008) PNAS 105: 4709-4714 (herein incorporated by reference in its entirety), were conducted to compare global gene expression patterns between two groups of cells comprising the following five lines of iPSCs (CCD1079-iPS, hNPC-iPS_{—}8, hNPC-iPS_{—}9, hNPC-iPS_{—}10 and IMR-90-iPS) and three types of parental somatic cells (IMR-90, hNPC and CCD1079).

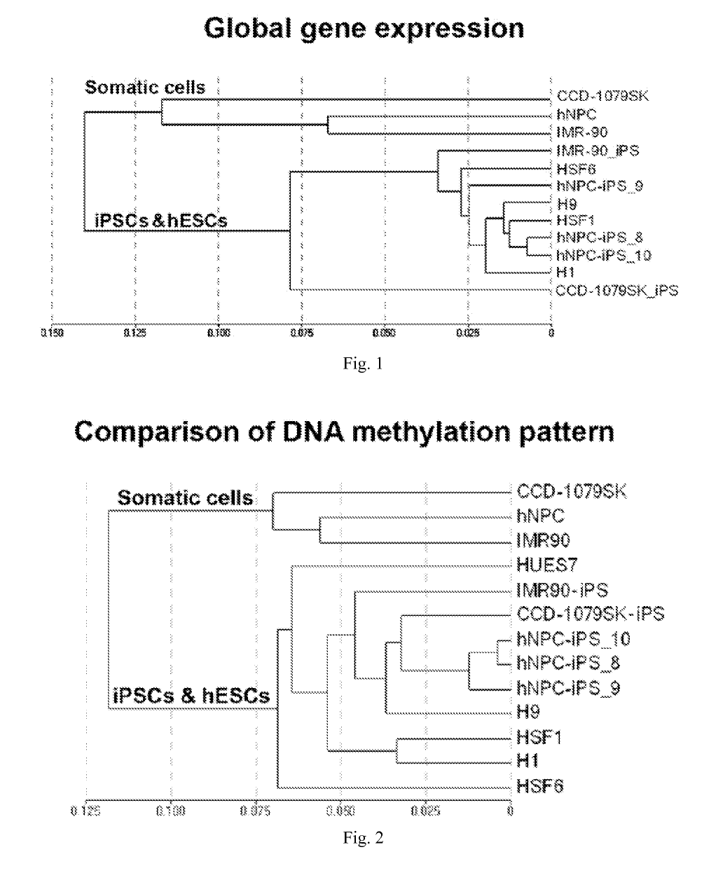

Four lines of hESCs (H1, H9, HSF1, and HSF6) generated from two different labs were used as control (NIH codes, WA01, WA09, UC01, and UC06 (Abetya et al. (2004) Human Molec. Genetics 13:601-608; and Thomson et al. (1998) Science 282, 1145-1147), which are herein incorporated by reference in their entirety). Whereas, NPC-iPSCs and CCD1097SK-iPSCs were generated with Oct4, Sox2, Klf4, and c-Myc (OSKM) retroviruses (Takahashi et al. (2007) Cell 131:861-872, herein incorporated by reference in its entirety), the IMR90-iPSC cell line was separately generated with retroviruses expressing Oct4, Nanog, Klf4, and Lin28 ((ONKL) (Yu et al. (2007) Science 318:1917-1920, herein incorporated by reference in its entirety). Consistent with previous findings, unsupervised clustering analysis of genome-wide gene expression revealed that all human iPSCs and hESCs are grouped together, whereas three types of somatic cells are clustered into their own distinctive branch. See Takahashi et al. (2007) Cell 131:861-872; Yu et al. (2007) Science 318:1917-1920; Park et al. (2008a) Cell 134:877-886; Park et al. (2008b) Nature 451:141-146; and Lowey et al. (2008) PNAS USA 105:2883-2888, which are herein incorporated by reference in their entirety. Thus, regardless of initial source of somatic cells or defined transcription factors in reprogramming, human iPSCs exhibit an overall hESC-like gene expression pattern. See FIG. 1.

### Genome-Wide Methylation of Promoter CpG Islands

After confirming that human iPSCs are highly similar to hESCs in morphology and in gene expression profiles, whether the global gene promoter methylation pattern of iPSCs resembles that of hESCs, or their parental somatic cells, or a mixture of the two was examined. The methylation states of 26,837 CpGs of 14,152 genes were profiled with INFINIUM HUMANMETHYLATION27 BEADCHIP microarrays (Illumina, San Diego, Calif.). The INFINIUM HUMANMETHYLATION27 BEADCHIP microarray is primarily designed to interrogate methylation in the promoter region (93.5% of all CpGs within the proximal promoter, which is defined as the region±1 kb of the transcription start site); moreover, a majority of CpGs (72%) are within CpG islands with the average coverage of approximately two CpGs per gene. Methylation profiling was performed in technical duplicates that exhibited very high correlations (average R²=0.998). Hierarchical clustering analysis of methylation patterns in these 26,837 CpGs demonstrated that the overall gene promoter methylation pattern of iPSCs is highly similar to that of five lines of hESCs (H1, H9, HSF1, HSF6, plus HUES7, see Cowan et al. (2004) New Engl. J Med. 350:1353-1356, which is herein incorporated by reference). See FIG. 2. In contrast, the parental somatic cells have distinctly different promoter methylation pattern from both iPSCs and hESCs.

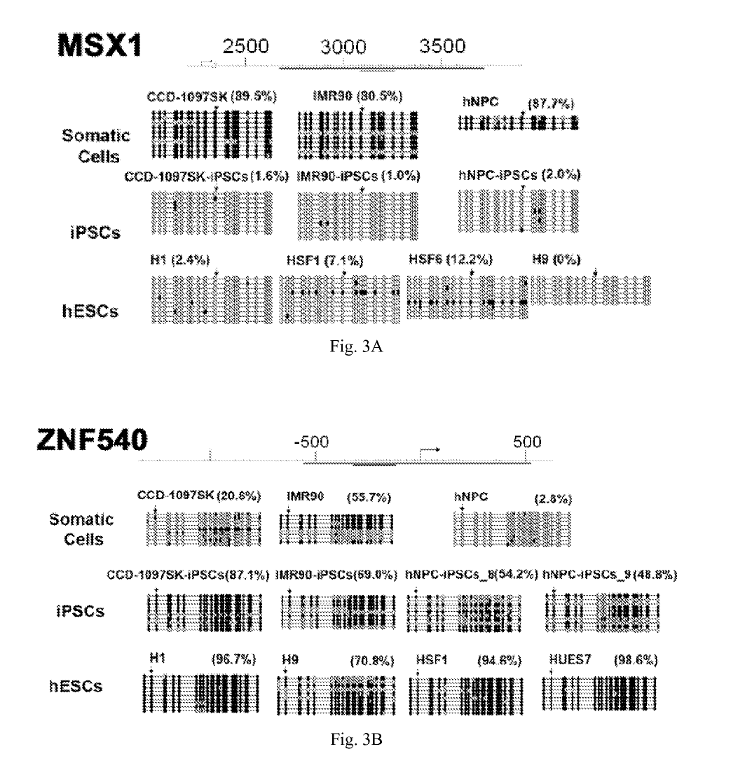

Bisulfite genomic sequencing analysis was performed to validate the microarray results using methods known in the art. See Bibikova et al. (2006) Genome Res. 16:1075-1083; and Shen et al. (2006) Human Molec. Genetics 15:2623-2635, which are herein incorporated by reference in their entirety. As shown in FIG. 3A and FIG. 3B, bisulfite sequencing was carried out to measure methylation in the representative CpG islands with one in the gene promoter region and the other in the gene body. It is noted that iPSCs and hESCs exhibit similar methylation patterns in both MSX1 and ZNF540 CpG islands.

The results confirmed that there was a significant increase of methylation in the ZNF540 gene promoter and a significant reduction of methylation in the gene body of MSX1 in all iPSC lines when compared to their parental somatic cells. In contrast, although different lines of iPSCs and hESCs exhibited certain variations in their methylation levels, the overall methylation patterns between all iPSCs and hESCs are very similar in these two CpG islands. See FIG. 3A and FIG. 3B.

Promoter CpG Methylation Between iPSCs and Their Parental Somatic Cells

Because three types of somatic cells are derived from different tissue origins and developmental stages, each cell type has its own cell-specific methylation pattern (data not shown). The methylation levels in each CpG between iPSCs and their parental somatic cells were compared using INFINIUM HUMANMETHYLATION27 BEADCHIP microarrays. The genes that exhibit statistically significant changes in CpG methylation patterns (with the delta-beta value≧0.3 or ≦−0.3) were identified (data not shown).

This data indicates that approximately 7-14% of gene promoters undergo methylation changes during direct reprogramming. 86-93% (i.e. 93% NCP-iPSCs, 91% IMR90-iPSCs, 86% CCD-1097SK-iPSCs) CpGs showed no significant difference in methylation. Surprisingly, the number of gene promoters exhibiting an increase in methylation was found to be 3.5-6 fold more than the number of gene promoters showing a decrease in methylation. Approximately 6-11% (i.e. 6% NCP-iPSCs, 7% IMR90-iPSCs, 11% CCD-1097SK-iPSCs) of genes showed an increase methylation in iPSCs when compared with parental somatic cells, whereas only 1-3% (i.e. 1% NCP-iPSCs, 2% IMR90-iPSCs, 3% CCD-1097SK-iPSCs) of genes exhibited a decrease in DNA methylation.

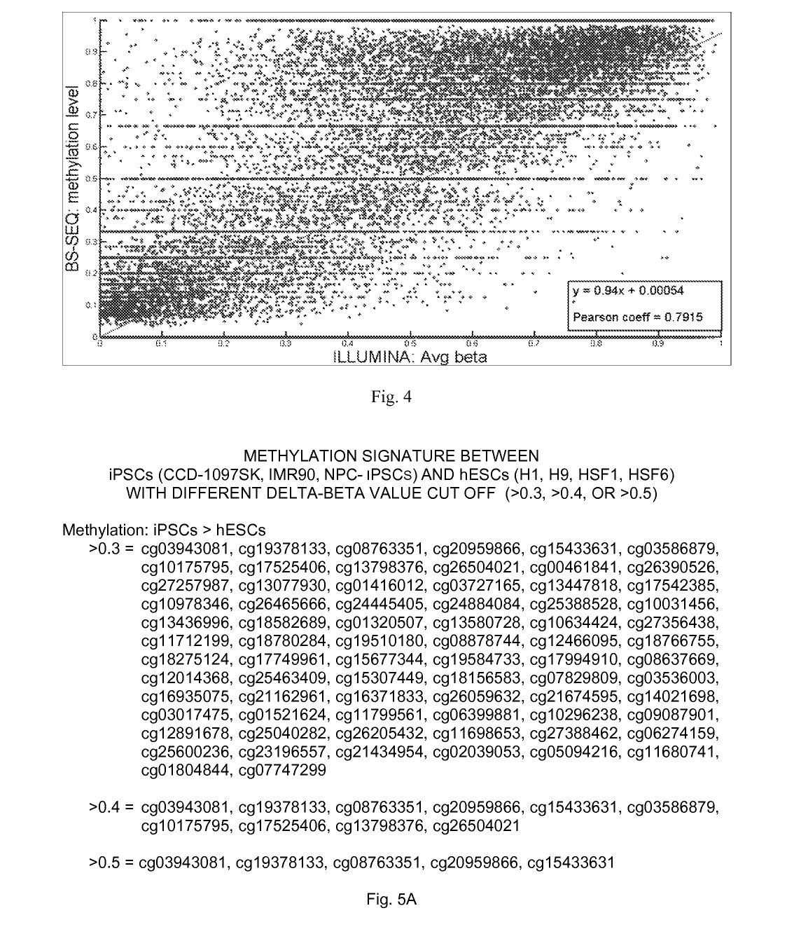

Methylation changes in paired somatic cells, iPSCs and hESCs by bisulfite sequencing was validated. In particular, as provided in FIG. 4, the average beta (β) of each probe in H1 and IMR90 cells was cross-referenced to deep genome-wide bisulfite sequencing data on the same cells generated by Lister et al. (2009) Nature 462:315-22, which is herein incorporated by reference in its entirety. Because both datasets use NCBI36 (hg18) build as the reference genome, the Illumina CpG targets were directly mapped to the Lister dataset and analyzed. The analysis shows a strong correlation between β and methylation level (Pearson coefficient=0.80). CpGs with low levels of methylation generally correspond to small β values, indicating that small β values cannot discriminate between lowly methylated CpGs from unmethylated CpGs. Since the β sensitivity cannot resolve partial methylation, CpGs with low methylation levels are treated as unmethylated CpGs. To optimize the methylation level threshold to declare a CpG as unmethylated or methylated, serial ROC curves of different thresholds were generated. Methylation level of 0.40 best optimizes the specificity and sensitivity to distinguish methylated and unmethylated CpGs. These results indicate the β values can better predict unmethylated CpGs (β<0.14, FDR=0.025) than methylated CpGs (β>0.36, FDR=0.1). Using these criteria, about 90% of the CpGs analyzed herein were validated with reasonable confidence.

Bisulfite sequencing confirmed de novo methylation during reprogramming in GFP25, ZNF354C, SULT1A1 promoter CpG islands and demethylation in iPSCs in promoter CpG islands of PAX8, CDX1, ALX4, and LRRF1P1 genes (data not shown). This validation data suggests that assays with the INFINIUM HUMANMETHYLATION27 BEADCHIP microarrays can reliably detect differential methylation levels among multiple samples; moreover, the methylation status of the arrayed CpGs is highly representative of the methylation status in the broader promoter regions and CpG islands. Importantly, the bisulfite sequencing analysis indicated that those genes showing an increase in DNA methylation in iPSCs appeared to de novo methylated, because a majority of these genes in somatic cells are virtually unmethylated. Taken together, these results suggest that both demethylation and de novo methylation are important for successful cell reprogramming.

In order to understand what kind of genes are subject to demethylation and de novo methylation in reprogramming, gene ontology (GO) analysis for each pair of iPSCs and parental somatic cells was performed. For de novo methylation, NPC-iPSC, IMR90-iPSC, and CCD-1097-iPSC lines were enriched for different GO terms. For example, de novo methylated promoters in NPC iPSCs are enriched for genes in cellular component and protease inhibitor activity whereas those in IMR90 iPSCs are enriched for genes function in receptor activity, sugar binding, and the like. The genes subject to de novo methylation in CCD-1097SK iPSCs belong to genes involved in defense response and immune system process. These results suggest that during reprogramming different types of somatic cells have a different spectrum of genes that undergo de novo methylation.

In contrast, gene ontology analysis indicated that similar GO terms are shared by those gene promoters that undergo demethylation during the conversion of three types of somatic cells into iPSC lines. GO terms for demethylated gene promoters are involved mainly in development process, cellular metabolic process and gene regulation.

Correlation of de Novo Methylation with Gene Silencing

From the above experiments, it was found that both waves of de novo methylation and demethylation take place in the direct reprogramming of somatic cells into iPSCs. The correlation of methylation changes with the global gene expression changes for each pair of somatic cells and iPSCs was then examined (data not shown). The data showed that demethylation of both developmental process genes and pluripotency genes are associated with gene activation. Indeed, demethylation in OCT4 and CDX1 gene promoters are correlated with the activation of these genes in our iPSCs (data not shown).

To examine whether de novo methylation is associated with gene silencing during the conversion of somatic cells into iPSCs, cross-reference analysis was conducted and those genes that exhibit an increase of DNA methylation but a reduction or no gene expression in each line of iPSCs when compared to parental somatic cells were identified. The data (not shown) indicated that approximately 60-75% of genes that are subject to de novo methylation showed a significant reduction of gene expression or no expression in iPSCs. Gene ontology analysis indicated that these silenced genes are enriched for the genes required for specific functions such as defense response, immune system process, and receptor activity. Consistently, these silenced genes are depleted from genes involved in housekeeping functions such as intracellular membrane organelle, cellular metabolic process, and regulation of transcription. This analysis suggests that de novo methylation activities contribute to the silencing of genes involved in specialized cellular function and differentiation pathways during the conversion of somatic cells into human iPSCs.

Profiles of Single CpGs Distinguish iPSCs from ESCs and Parental Somatic Cells

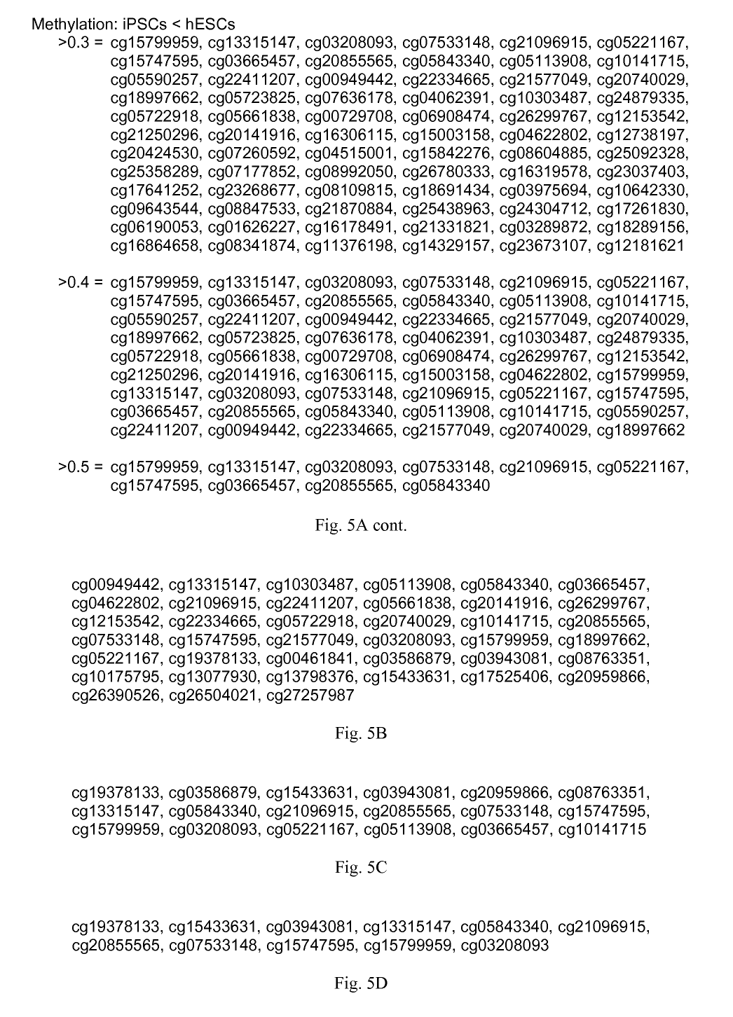

In the unsupervised clustering analysis of methylation in 26,837 CpGs over 14,152 genes, the five iPSC lines were found to be relatively clustered together. Then hierarchical clustering analysis was performed for differentially methylated genes between hESCs and iPSCs and between iPSCs and somatic cells. By using different stringencies of statistical analysis, it was found that the methylation profiles from as many as 175 CpGs in 146 genes (delta-beta>0.3, FIG. 5A), 39 CpGs in 31 genes (delta-beta>0.4, FIG. 5B) or as few as 18 CpGs in 15 genes (delta-beta>0.5, FIG. 5C) can effectively identify or distinguish iPSCs from either hESCs or parental somatic cells (data not shown). By further refining the clustering analysis, it was found that a minimum of 11 CpGs (FIG. 5D) in nine genes is sufficient to distinguish iPSCs and hESCs from each other. Significantly, these results show that these CpGs are uniquely methylated only in either iPSCs or hESCs, but not in somatic cells. Such a unique methylation profiles may be used to characterize human iPSCs and hESCs from each other.

To determine the biological significance of those genes that exhibit differential methylation between iPSCs and hESCs, Gene Ontology (GO) analysis was performed with the list of genes showing either a significant increase of methylation (delta-beta>0.3, CpGs n=84 in 74 genes) or decrease of methylation (delta-beta<−0.3, CpGs n=91 in 72 genes) in iPSCs when compared to hESCs. While significant GO terms for the pool of hypomethylated genes in iPSCs were not found, the analysis showed genes with more methylation (iPSCs>hESCs) are involved in epidermal cell differentiation, keratinization and tissue morphogenesis. This result suggests that hypermethylation of genes involved in tissue and cell differentiation contributes to the unique methylation pattern in iPSCs during cell reprogramming.

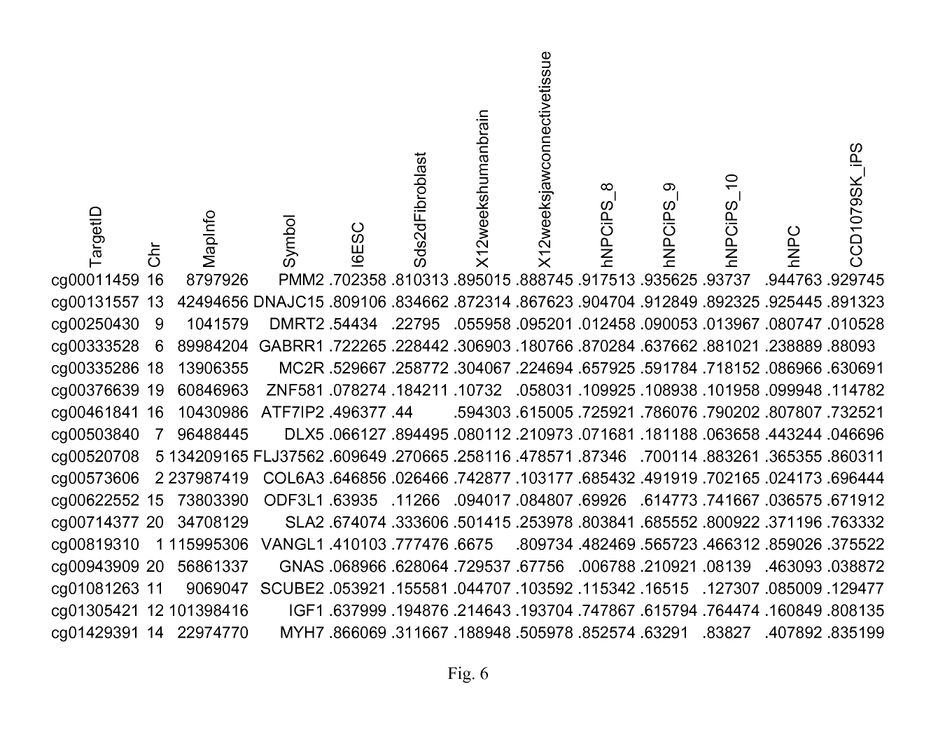

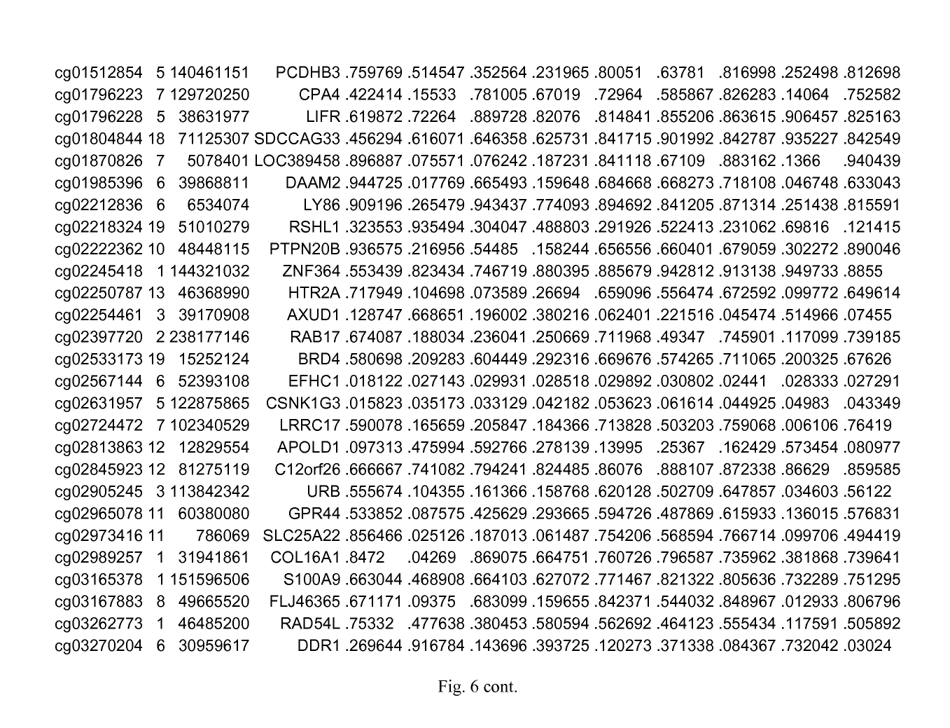

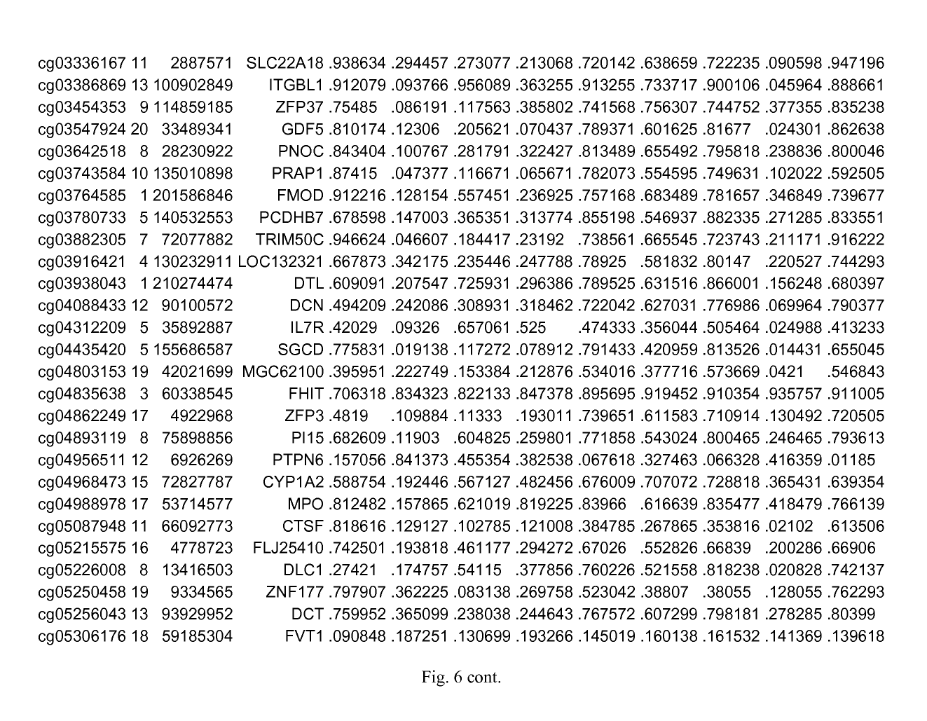

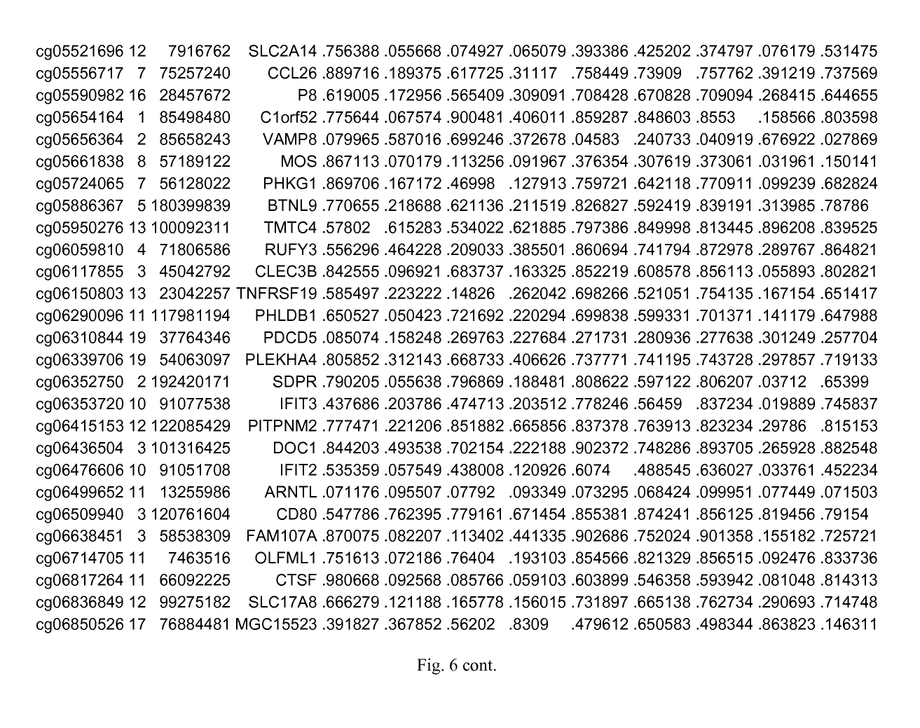

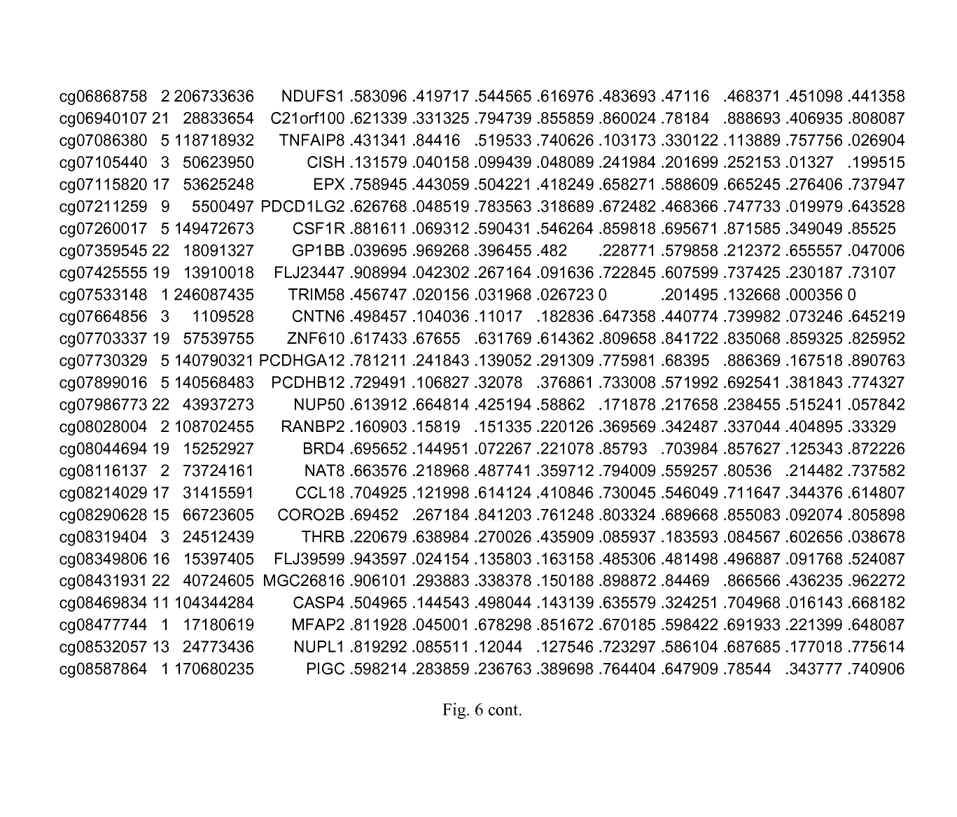

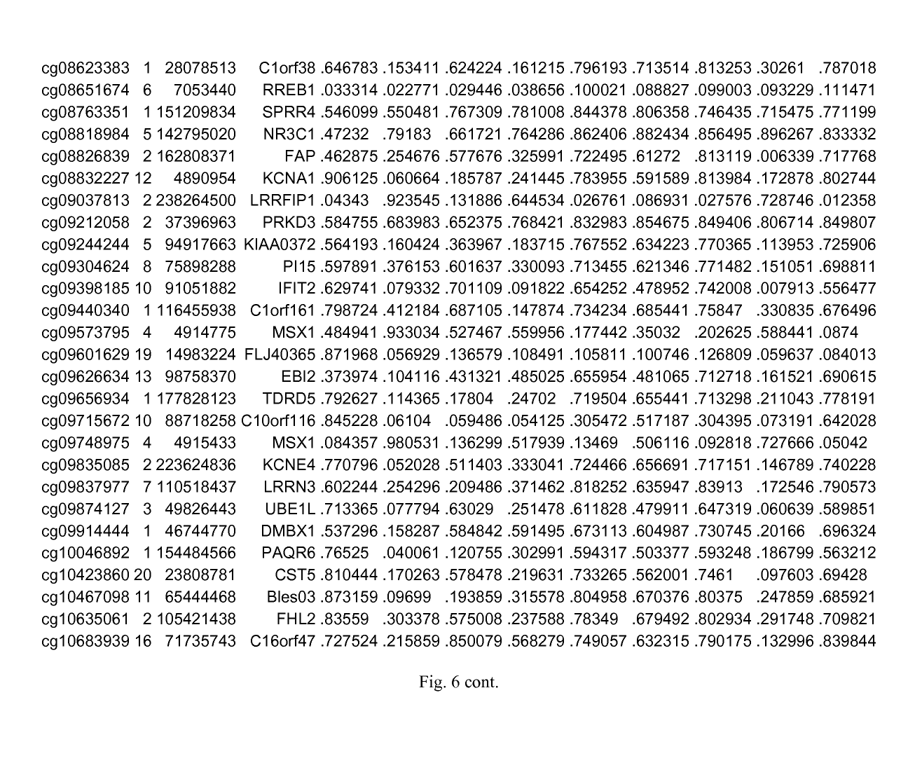

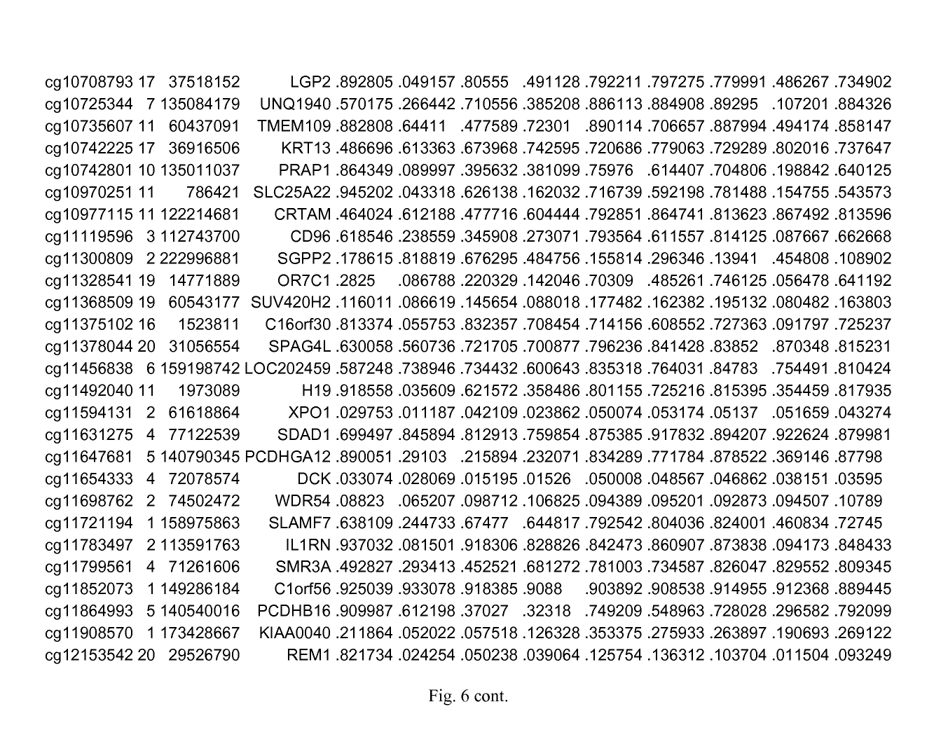

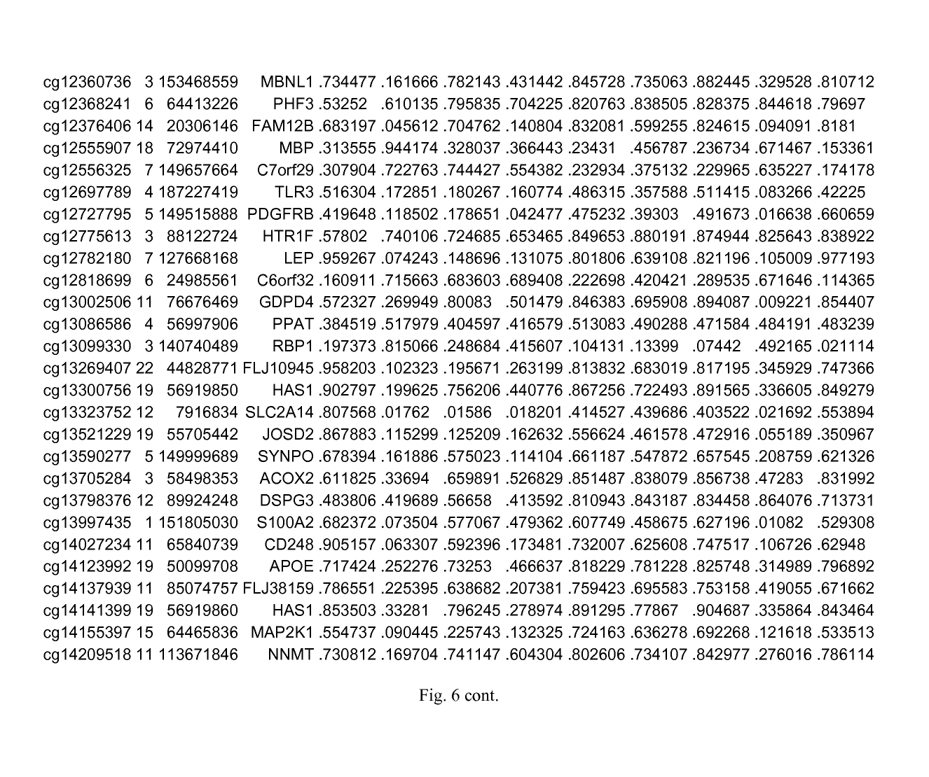

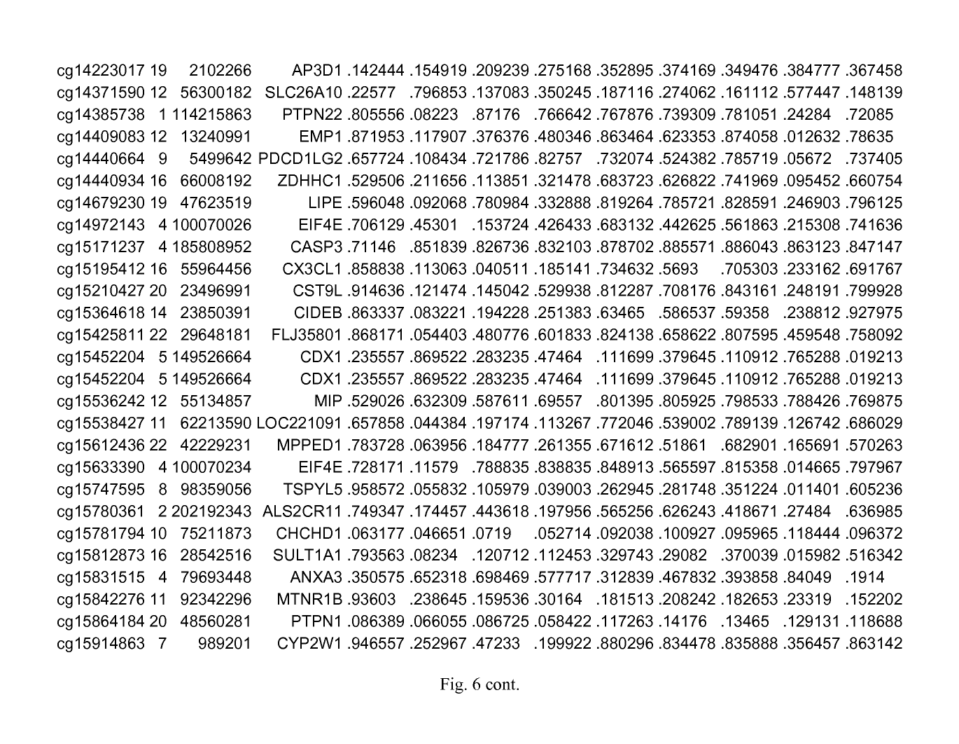

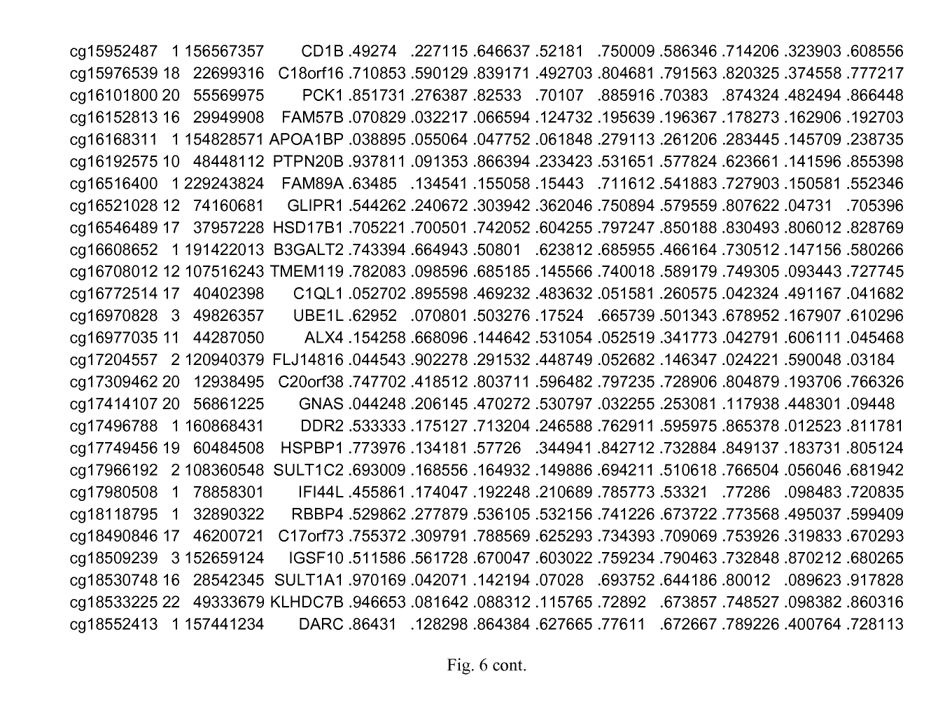

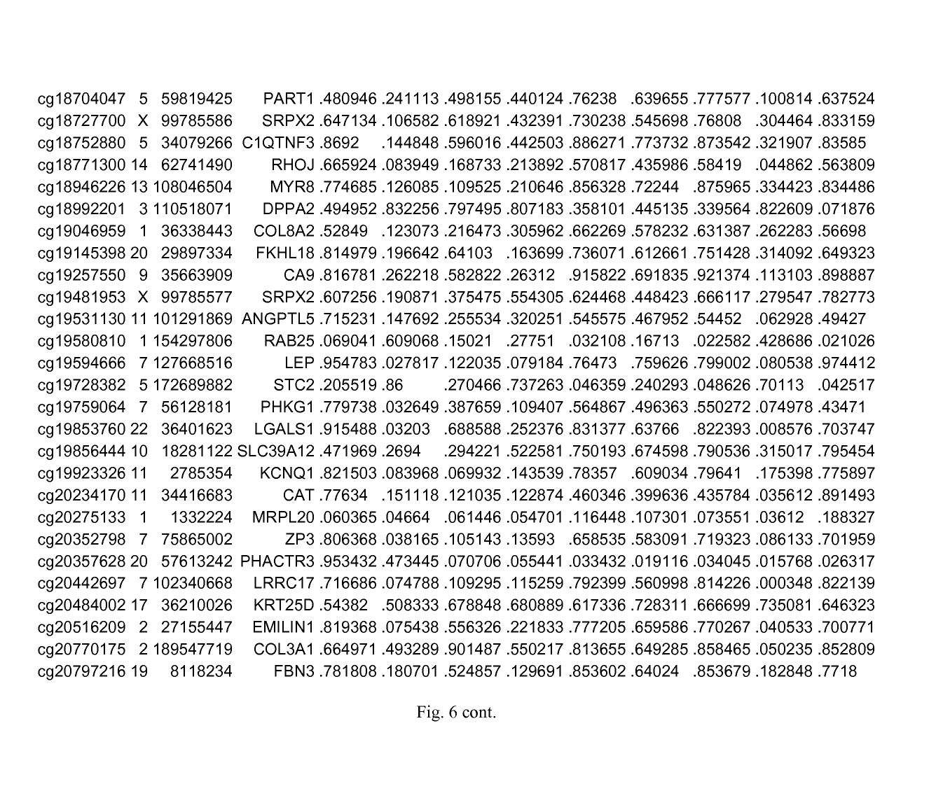

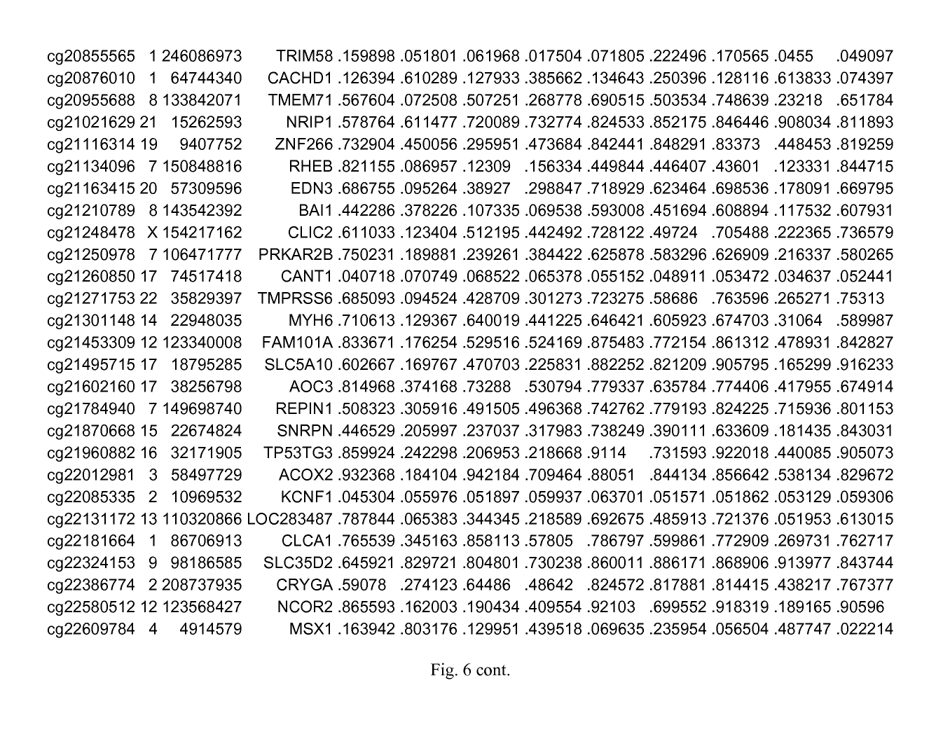

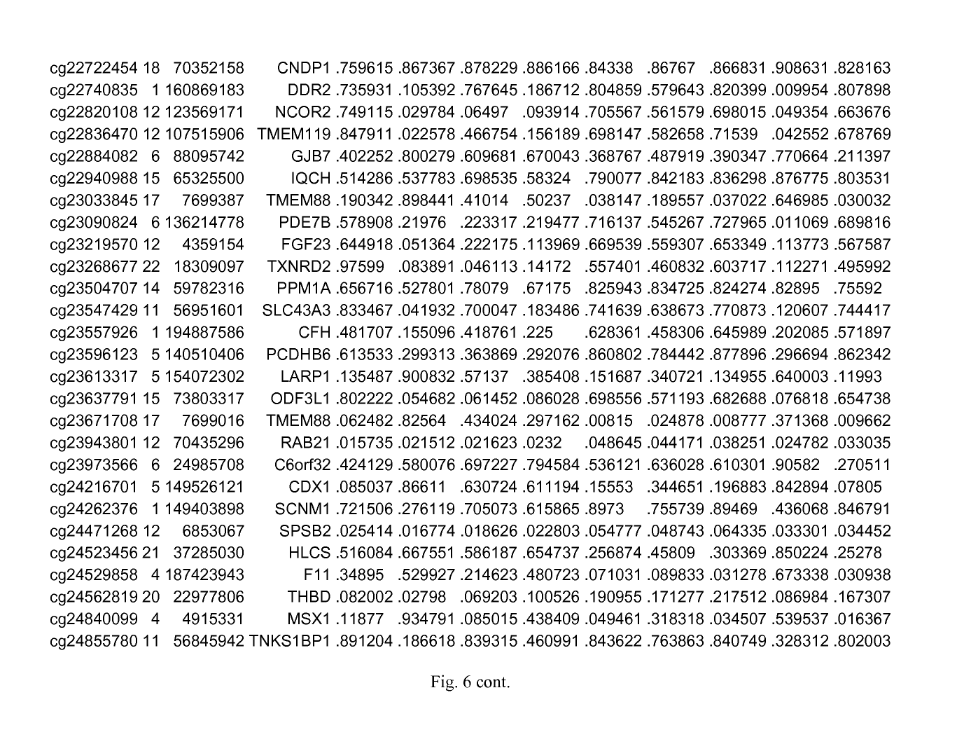

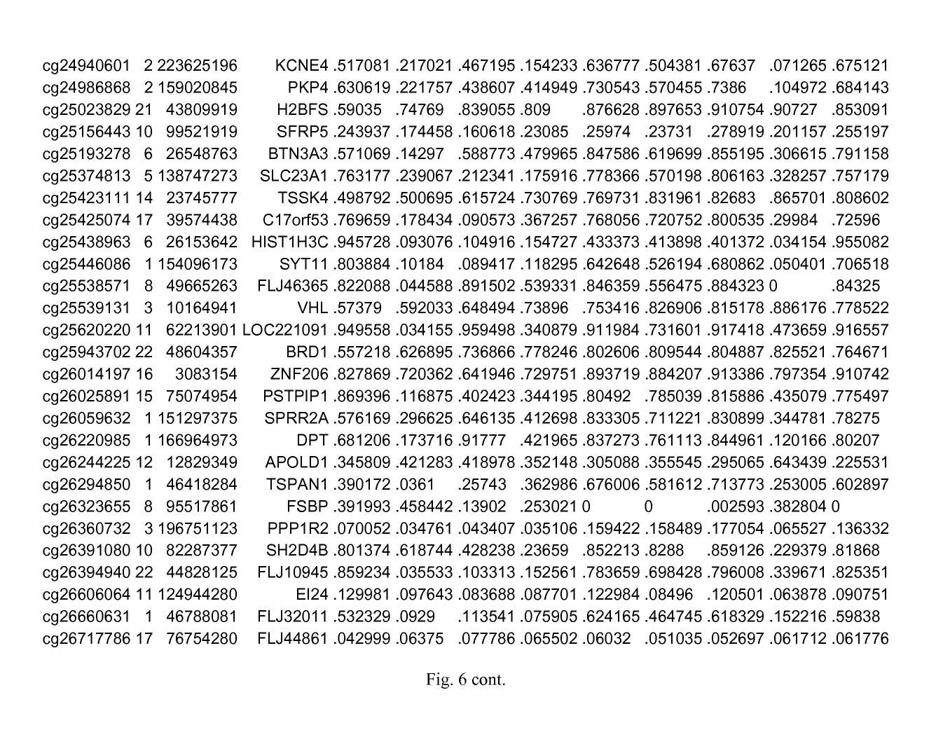

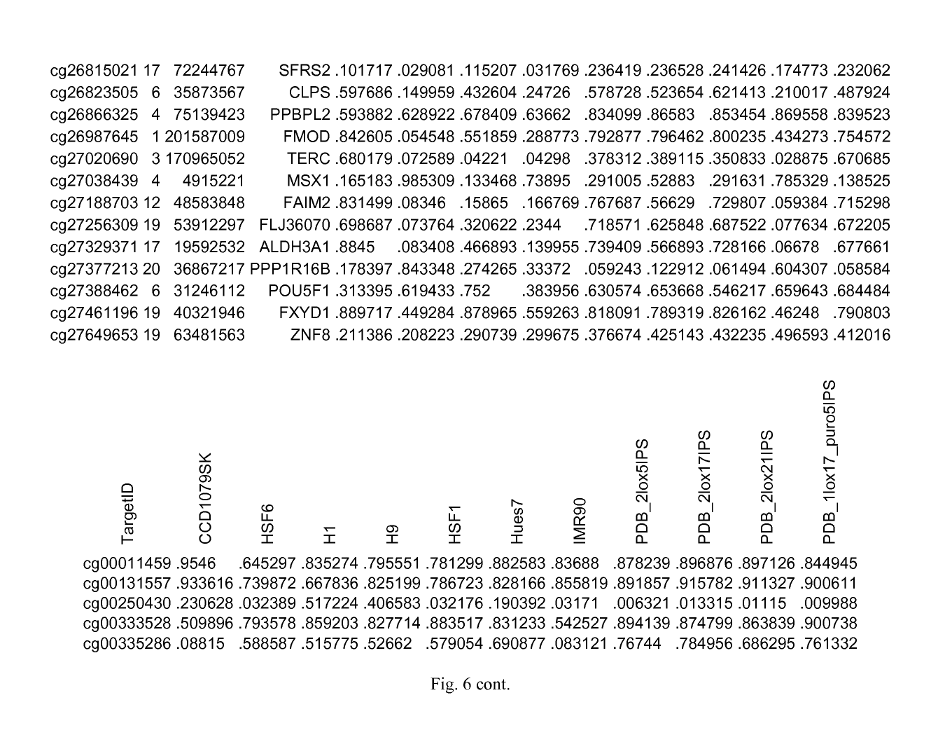

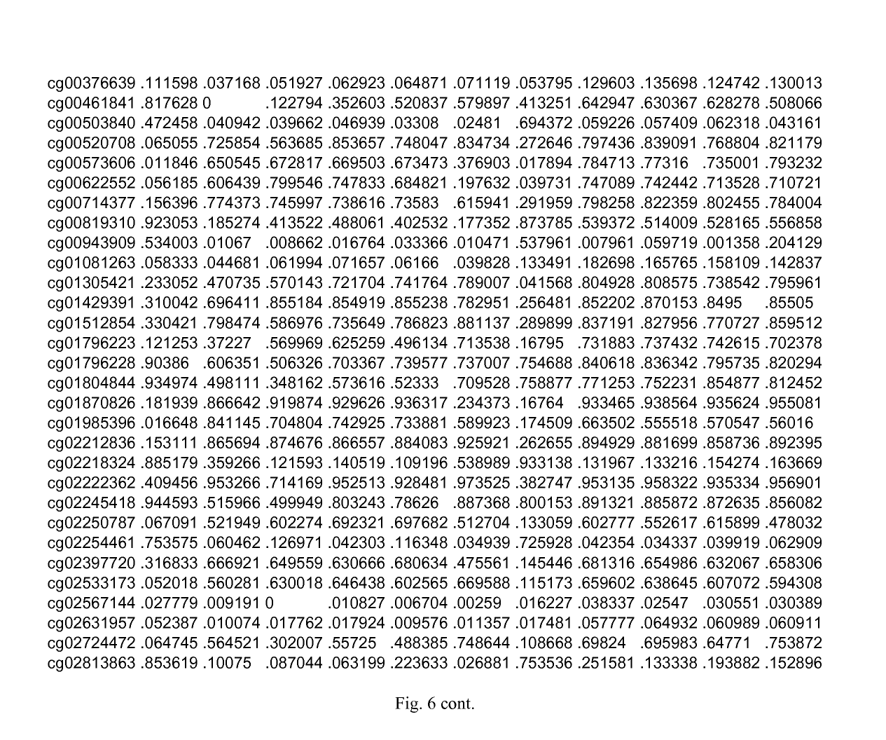

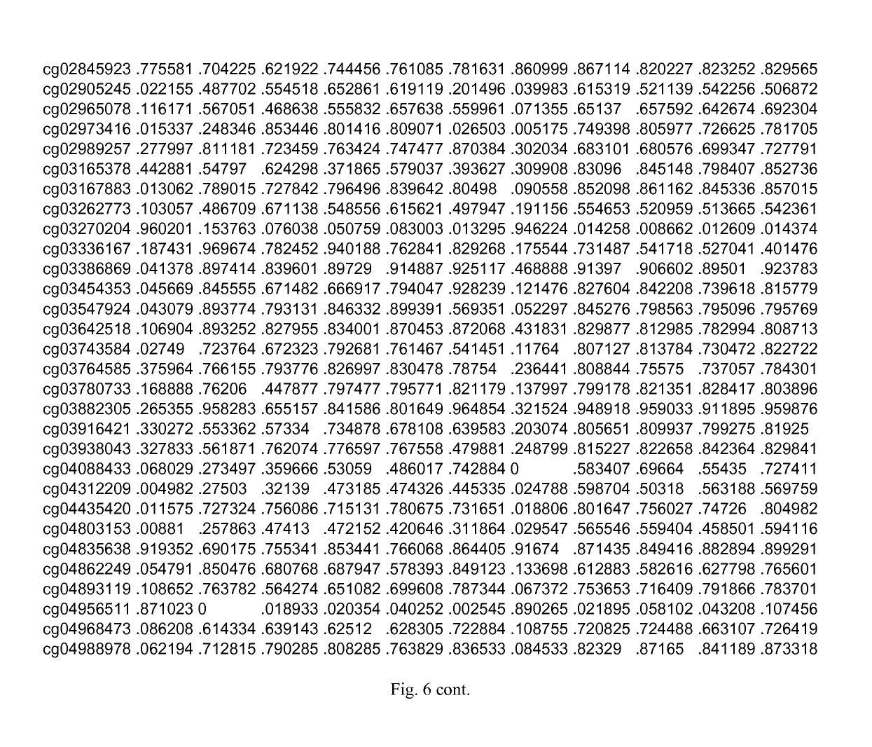

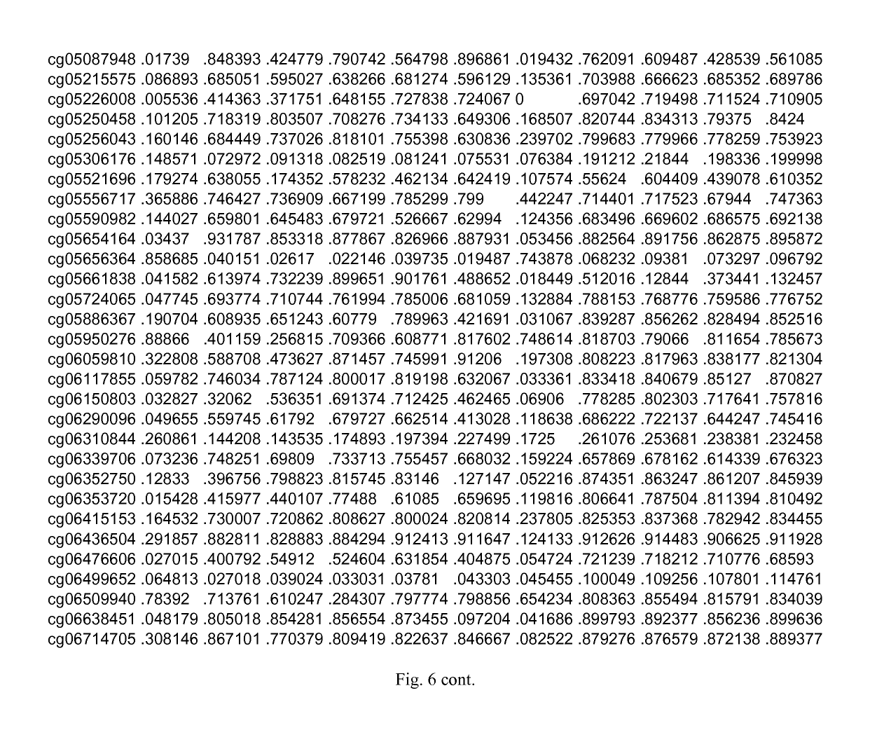

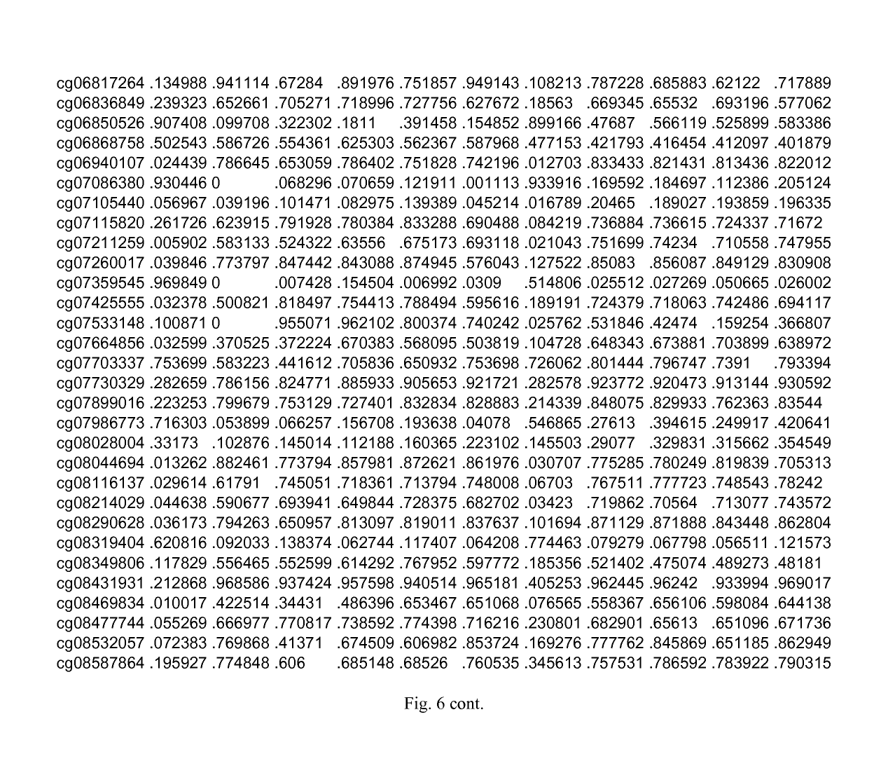

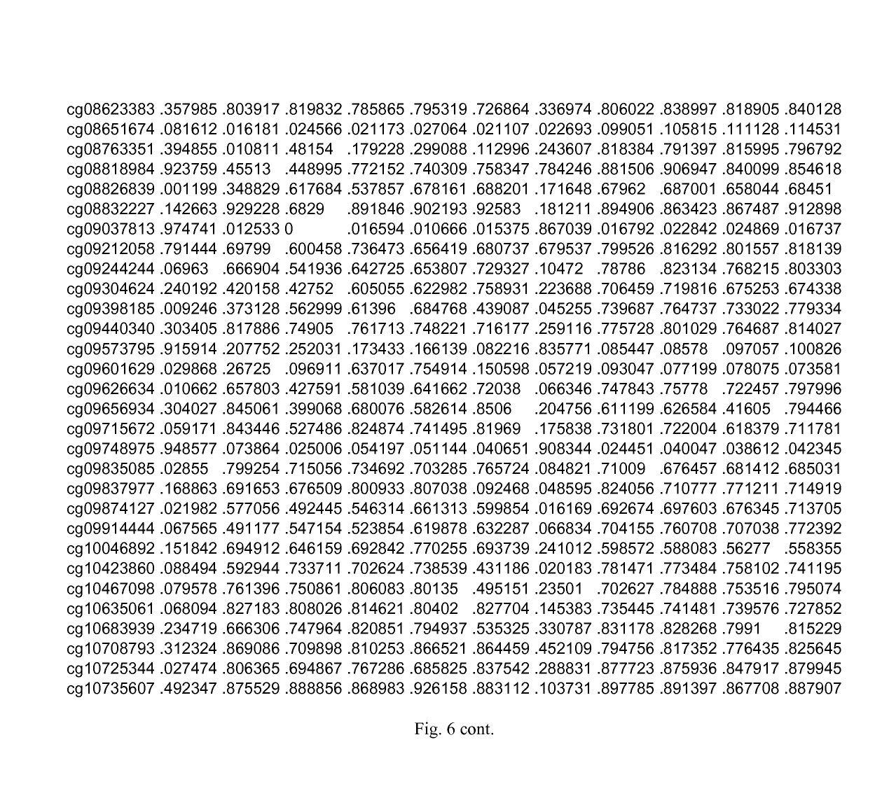

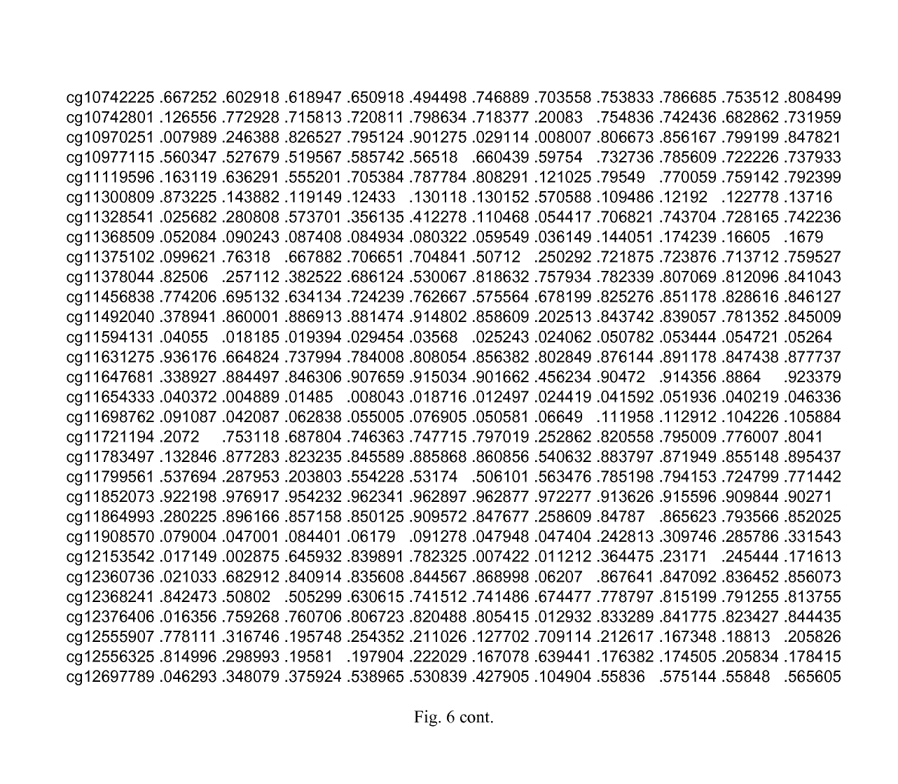

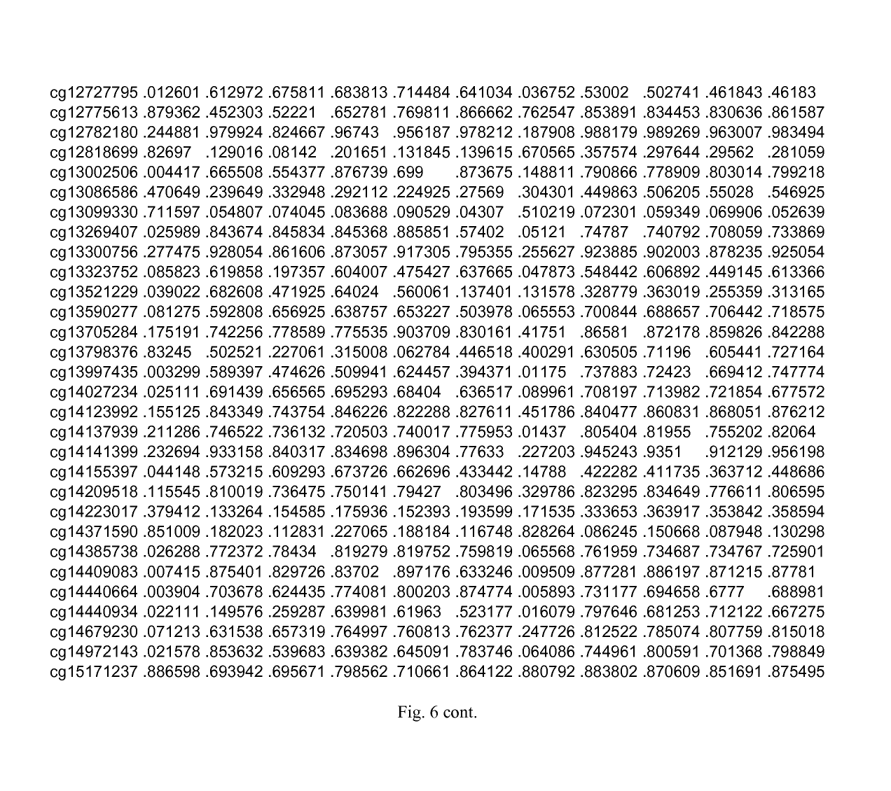

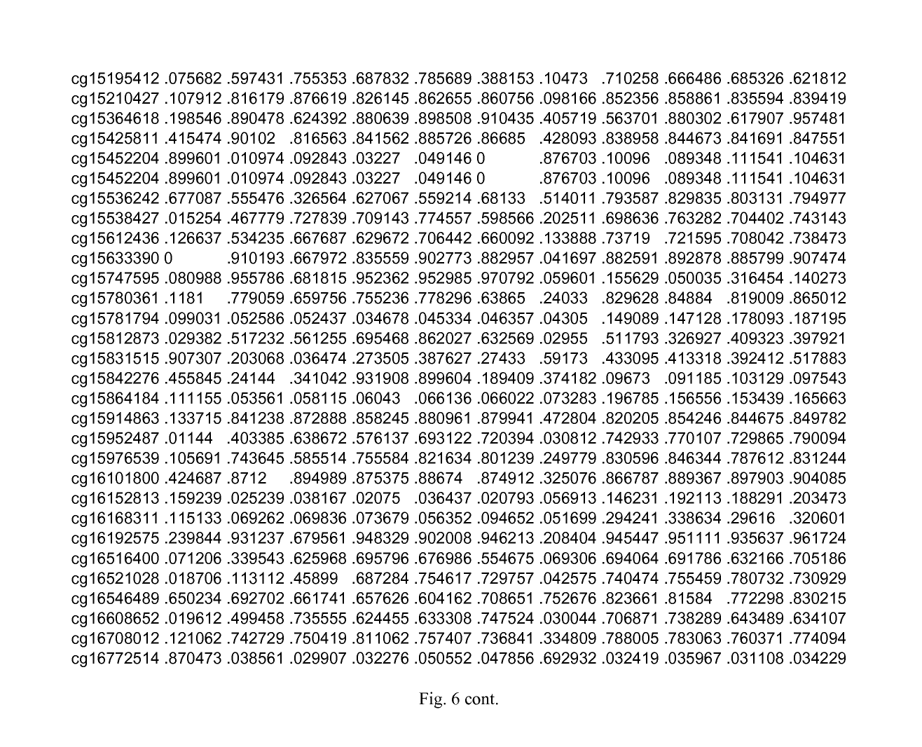

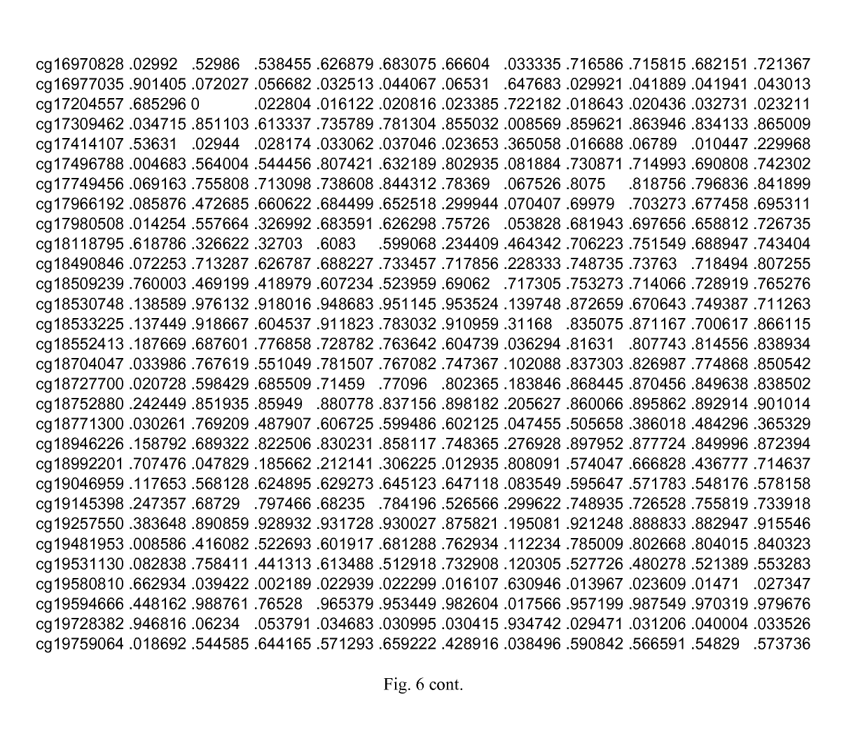

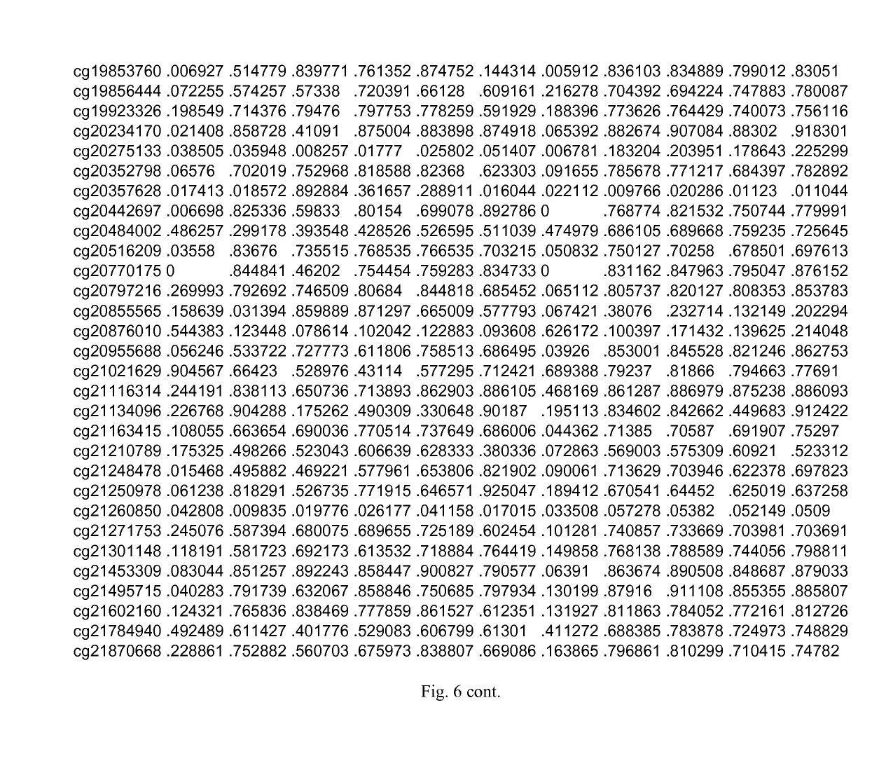

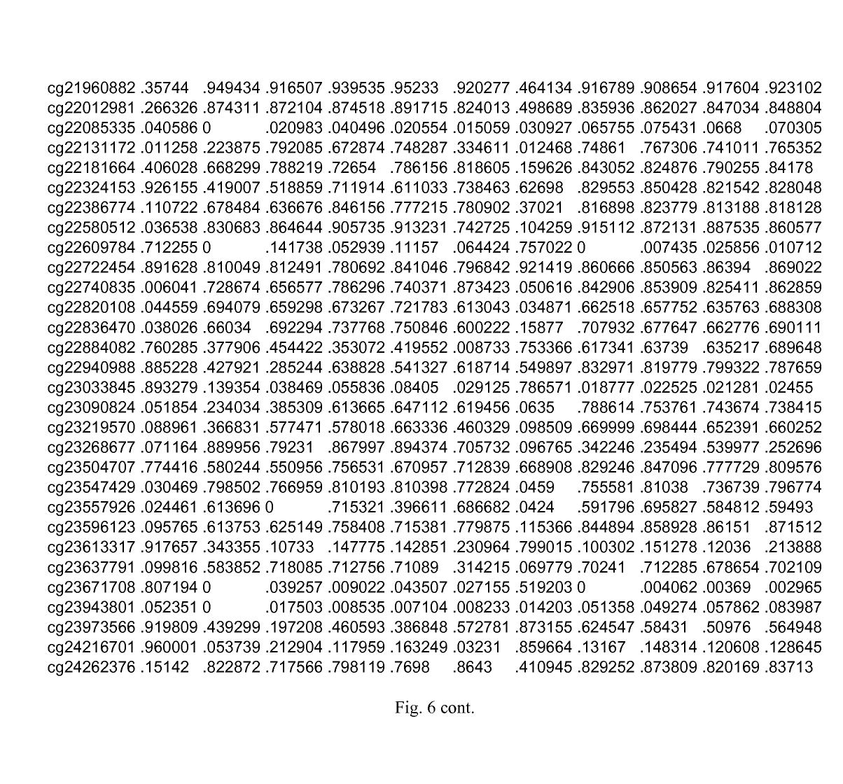

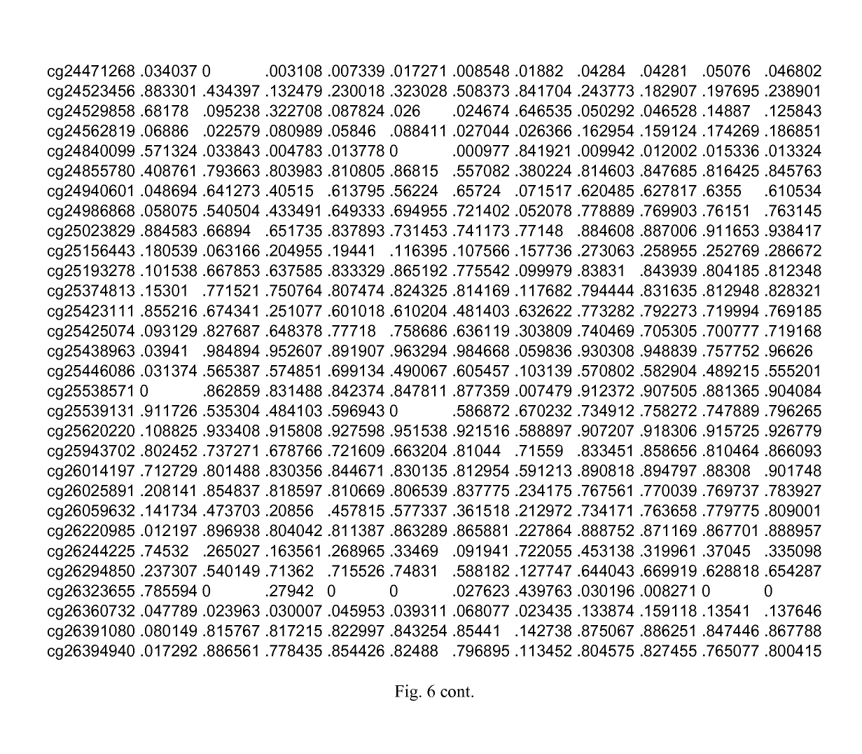

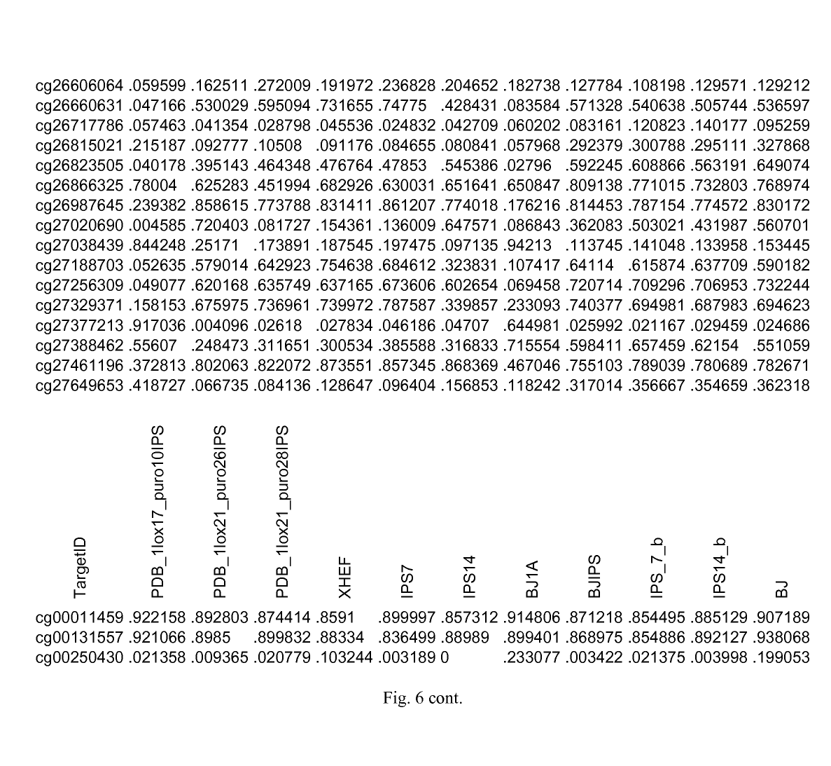

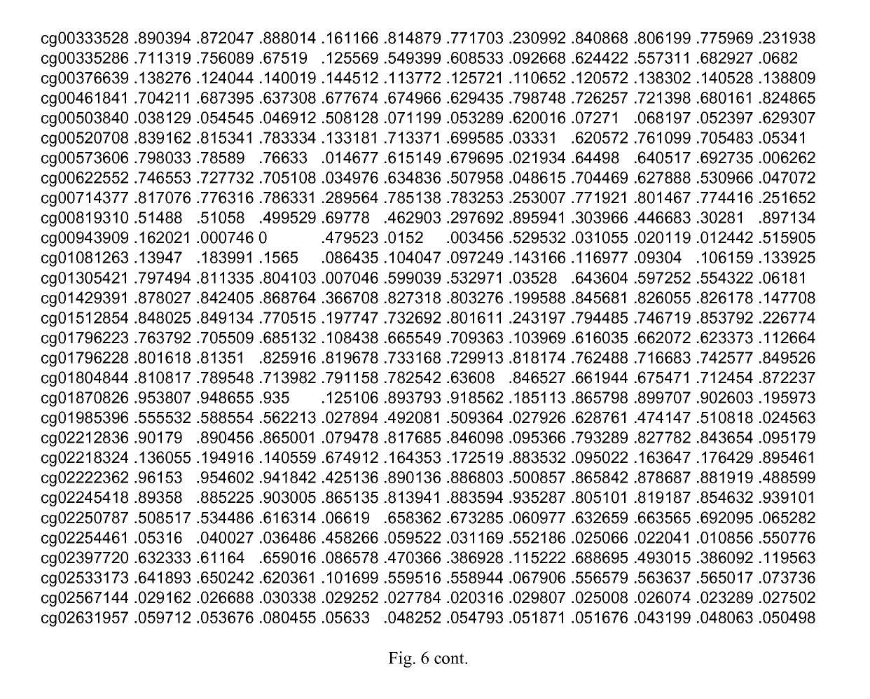

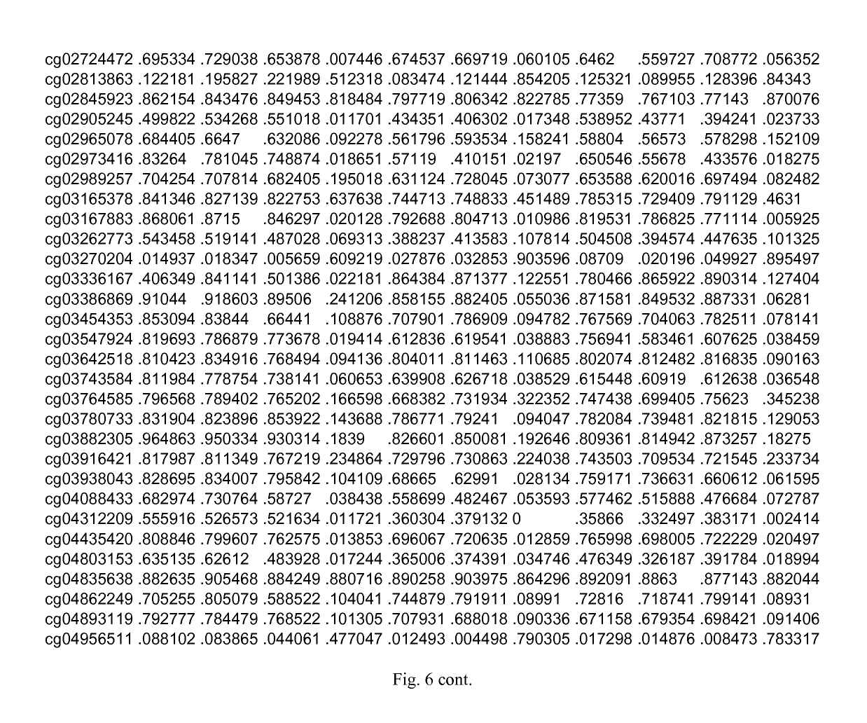

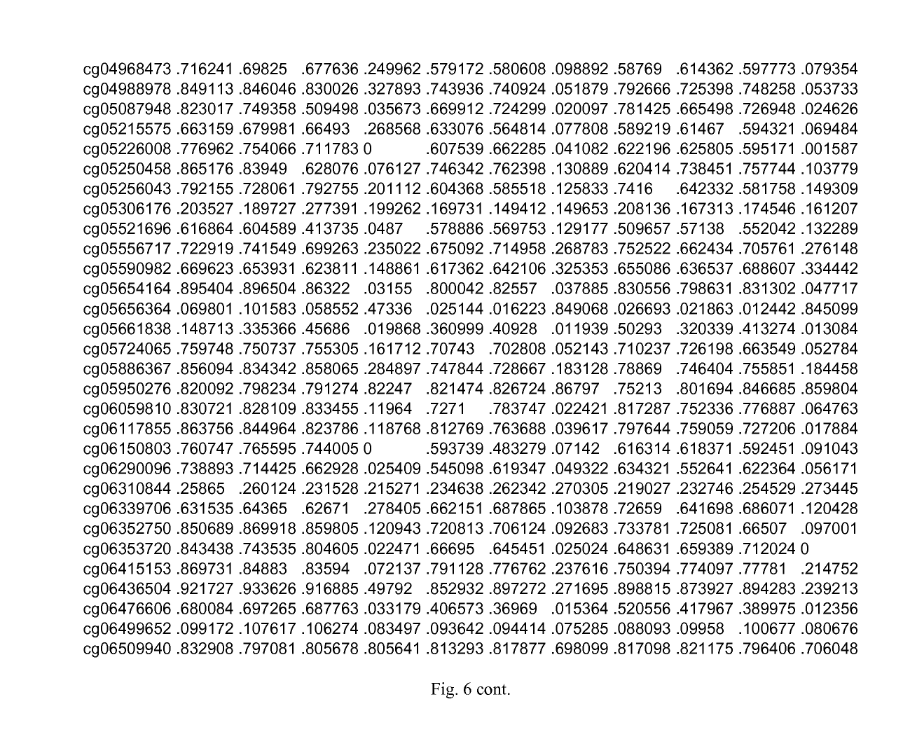

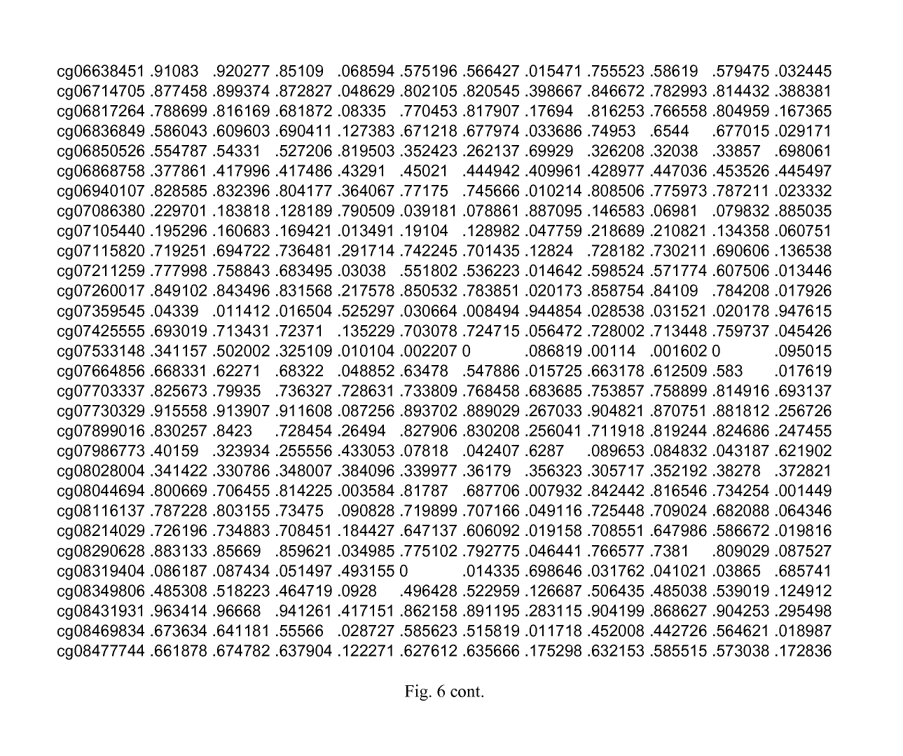

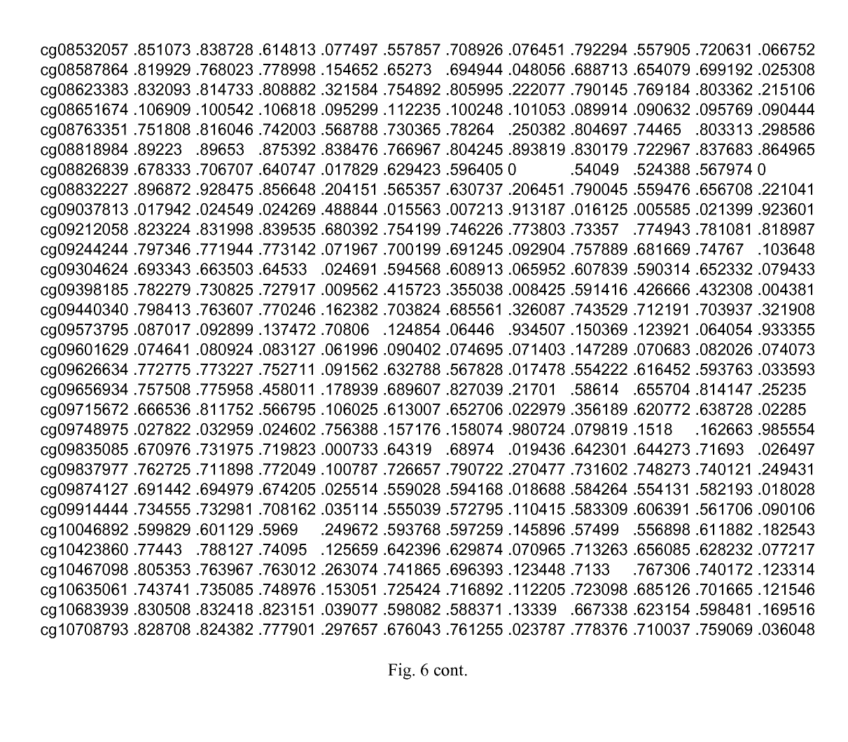

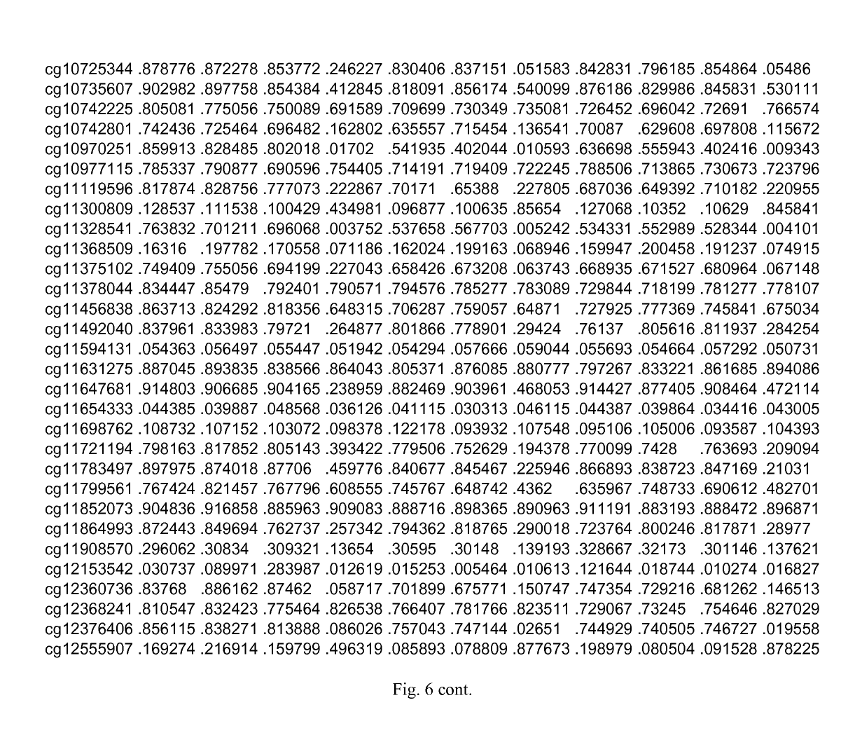

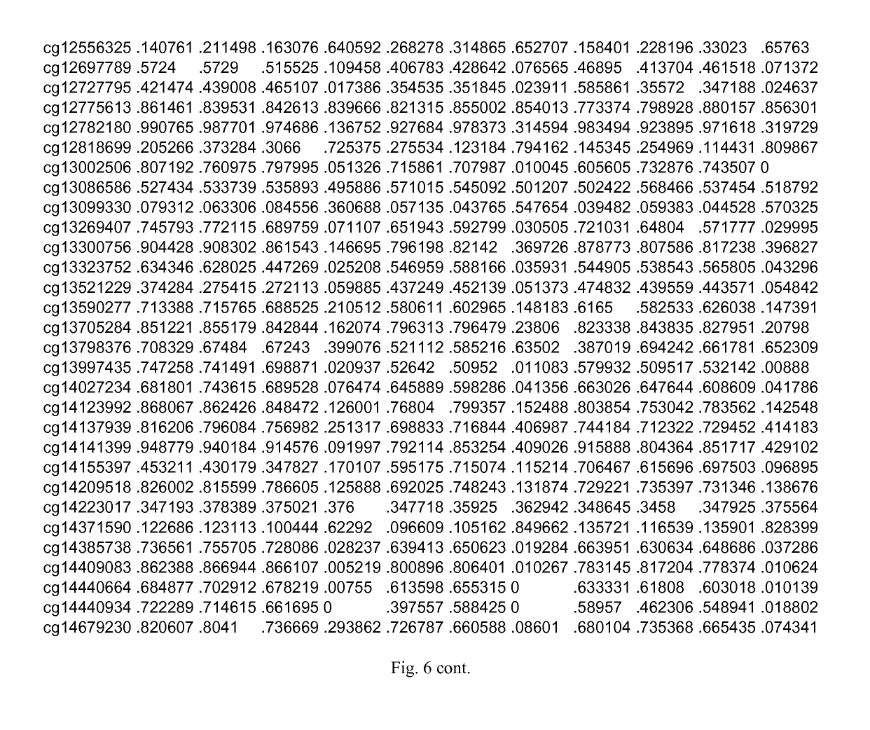

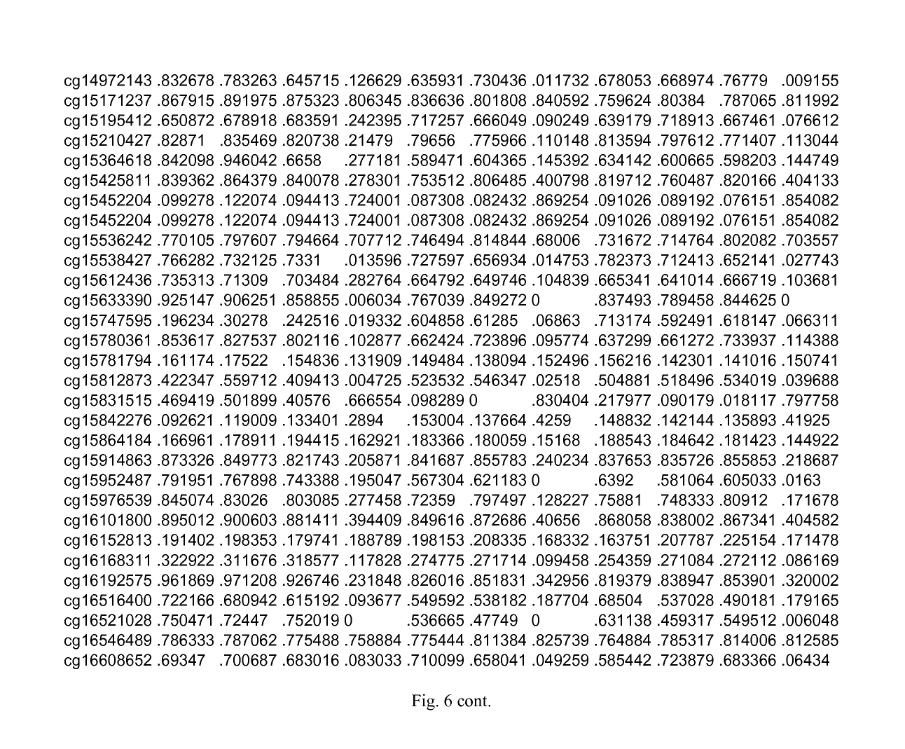

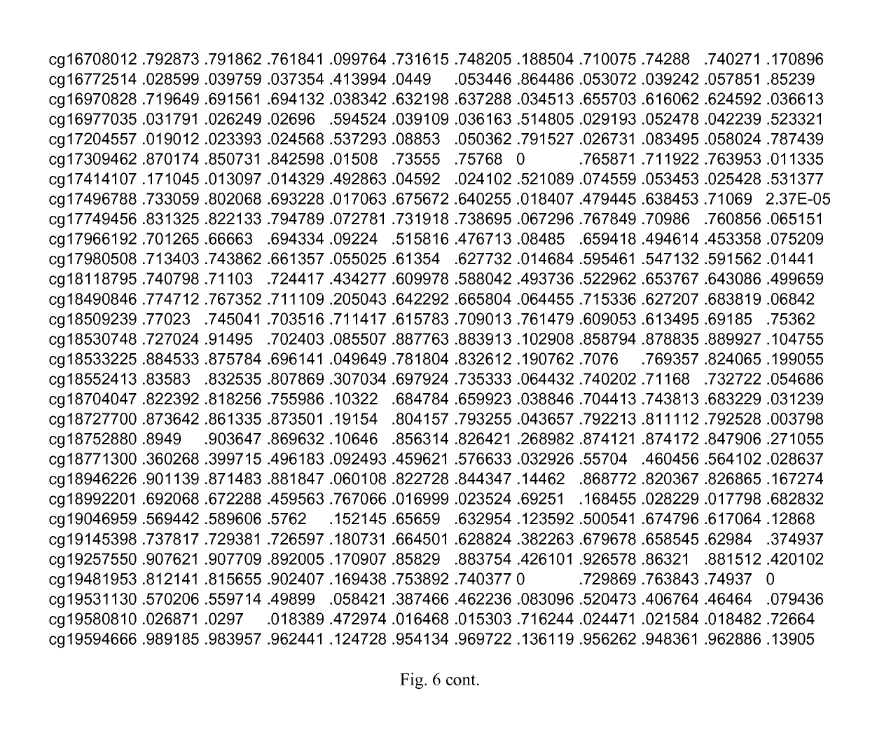

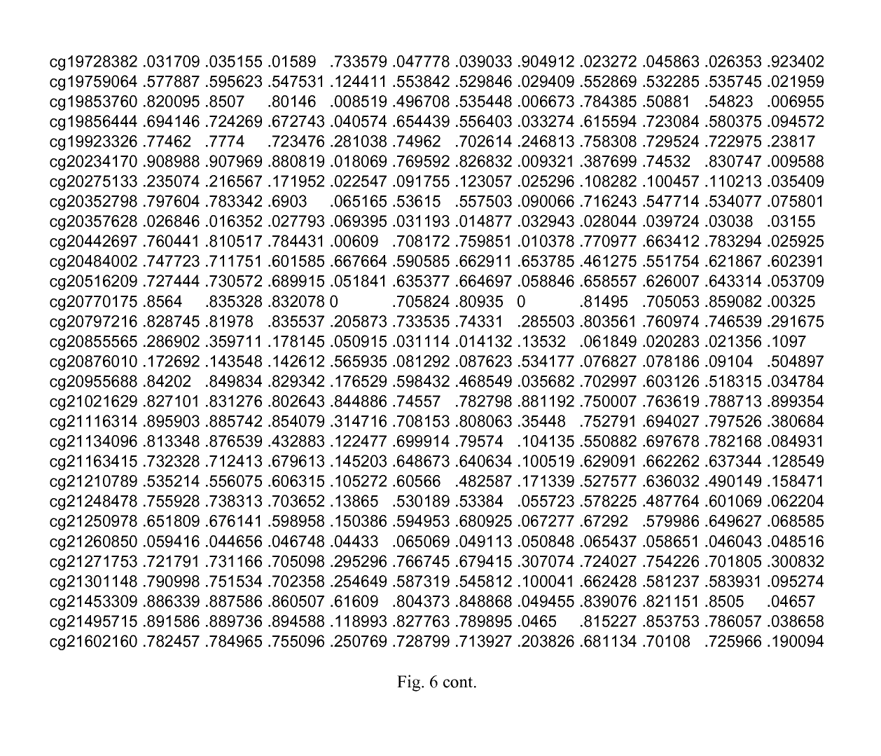

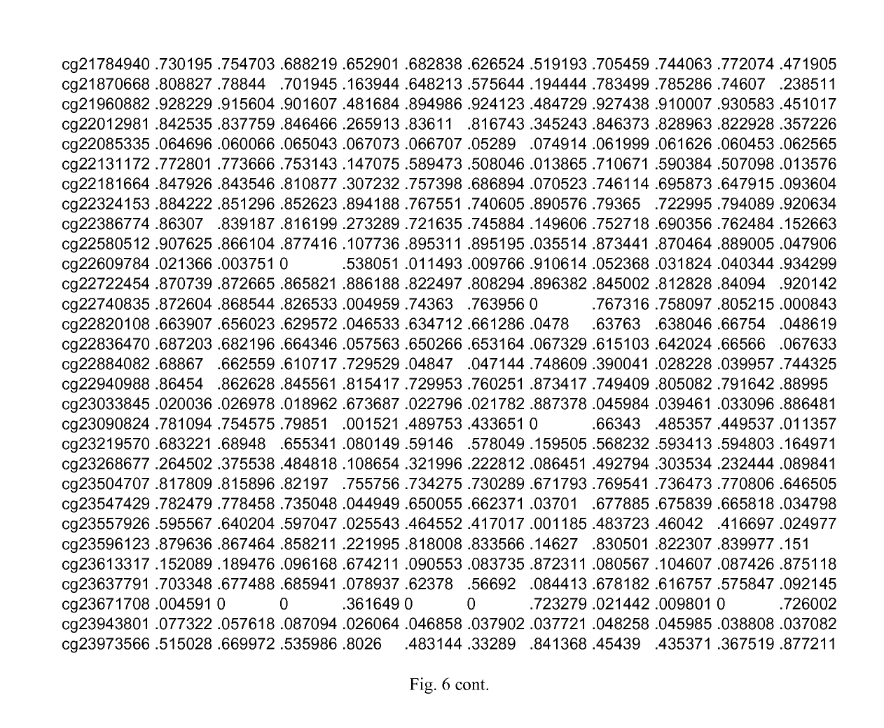

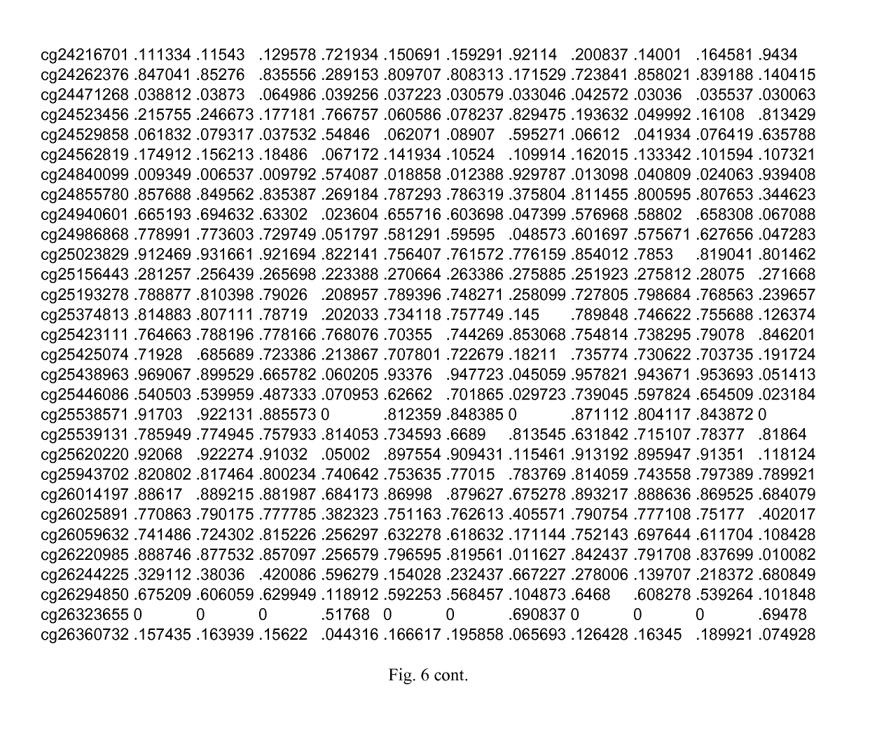

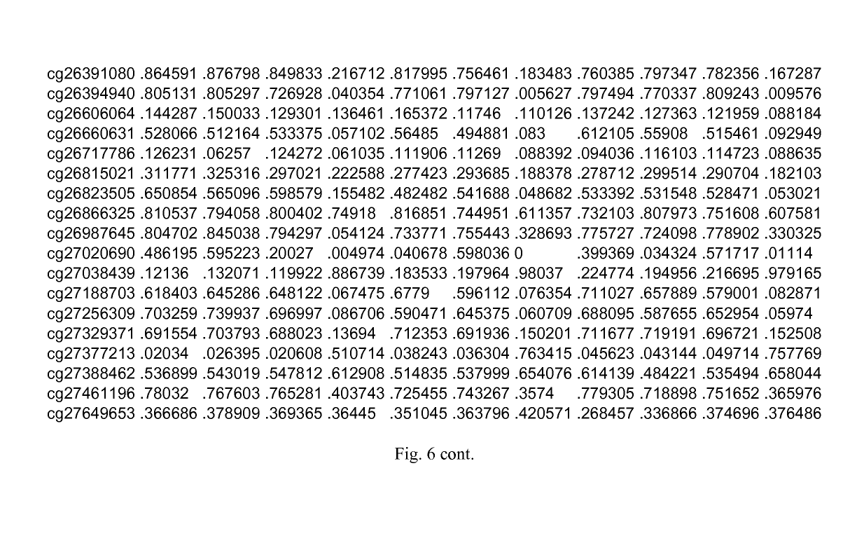

To examine whether differentially methylated genes exhibit differential gene expression in hESCs and iPSCs, real-time RT-PCR analysis of four representative genes, i.e. ZNF248, CYP2E, IRX2 and TCERG1, was performed. It was found that the methylation status is closely correlated with low level or no expression of these four genes in either iPSCs or hESCs (data not shown). Thus, differential methylation between iPSCs and hESCs is associated with differential gene expression in these cells. FIG. 6 is a table of all the CpGs, as exemplified herein, along with their associated human chromosome and map location and the beta values for various cell lines.

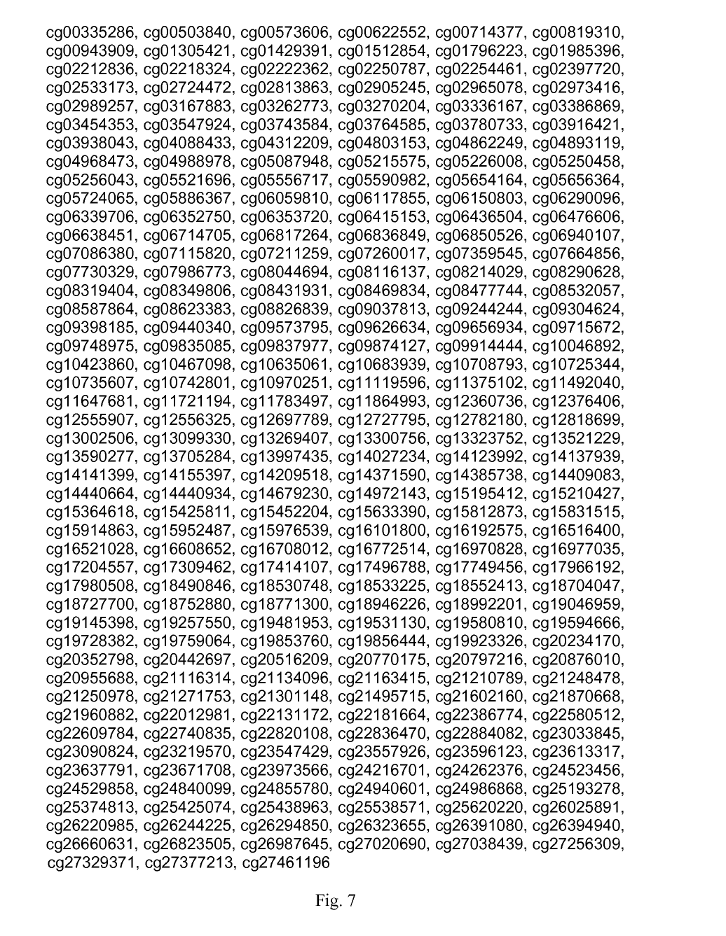

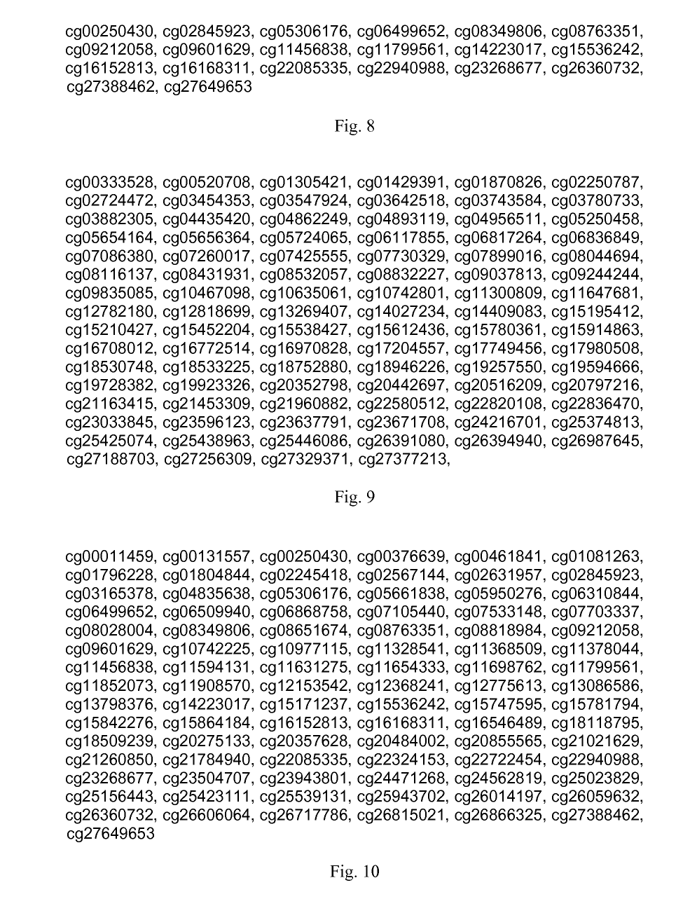

Additional cell lines and the methylation states of 273 CpGs were profiled with Illumina's INFINIUM HUMANMETHYLATION27 BEADCHIP (Illumina, San Diego, Calif.) microarray and analyzed. These 273 CpGs are listed in FIG. 7. FIG. 8 itemizes the CpGs which have statistically significant changes in the methylation patterns between the indicated iPSCs and the indicated ESCs. Statistical analysis indicates that the methylation profiles from 273 CpGs (delta-beta>0.3). FIG. 9 shows that 94 CpGs (out of the 273 CpGs with delta-beta>0.4, P<0.05, can effectively identify or distinguish somatic cells from iPSCs and hESCs.

To further identify methylation signature that can distinguish multiple cell types, the methylation markers (CpGs) that distinguish iPSCs from hESCs and somatic cells were determined. Interestingly, it was found that the profiles from as few as 91 CpGs may be used to effectively characterize a given cell (e.g. H1, HSF1, HSF6, HUES7, H9, I6, hNPC.iPS_{—}8, hNPC.iPS_{—}9, hNPC.iPS_{—}1, CCD.1079SK_iPS, IMR.90_iPS, PDB_{—}2lox.5IPS, PDB_{—}2lox.17IPS, PDB_{—}2lox.21IPS, PDB_{—}1lox.17_puro.5IPS, PDB_{—}1lox.17_puro.10IPS, PDB_{—}1lox.21_puro.26IPS, PDB_{—}1lox.21_puro.28IPS, IPS7, IPS14, BJ.IPS, IPS_{—}7_b, IPS14_b, hNPC, CCD.1079SK, IMR.9, XHEF, and BJ. FIG. 10 shows that the methylation profiles for these 91 CpGs for the indicated cell lines are distinct. Thus, as provided herein, the methylation states of individual CpGs may be used to characterize a cell as an ESC, an iPSC or a somatic cell. In addition, the methylation states of individual CpGs may be used to distinguish one cell (or cell line) from another cell, cell line or cell type.

These results are surprising in view of the research by Doi et al. See Doi et al. (2009) Nature Genetics 41(12):1350-1354. Doi et al. discloses CpGs associated with genes which overlap with genes to which some of the 91 CpGs disclosed herein are associated. For the CpGs of Doi et al. for some of these overlapping genes, Doi et al. indicates provides data which is inconsistent with the results disclosed herein. For example, Doi et al. indicates two CpGs of ZFP42 which show less methylation in iPSCs as compared to fibroblasts (somatic cells). However, as set forth in FIG. 6, for CpG ZFP42 (cg06274159), iPSCs (i.e. hNPC.iPS_{—}9, IMR.90_iPS) are more methylated than their parental somatic cells and no difference in methylation between BJ.IPS and its parental somatic cell. Similarly, as set forth in FIG. 6, ZFP42 (cg14189571) shows more methylation for hNPC.iPS_{—}8 as compared to its parental somatic cell and no difference in methylation for CCD.1079SK_iPS and IMR.90_iPS and their parental somatic cells, respectively. As another example, Doi et al. indicates that for the CpGs of ZNF248, iPSCs are more methylated than somatic cells. However, as set forth in FIG. 6, there is no observable difference in methylation for the ZNF248 CpGs, cg03208093 and cg15799959, between iPSCs and their parental somatic cells. In addition, Doi et al. does not disclose any information which would enable one to distinguish a given cell (or cell line) from another cell type or cell line. Further, it is noted that the CpGs as set forth herein and the CpGs of Doi et al. (associated with the overlapping genes, i.e. TCERG11, ZFP42, ASCL2, CYP26C1, MOS, PXMP4, ZNF248, CD248, COL1A1, COL6A3, DAAM2, FAM26B, MBNL1, MC2R, MCF2L, NCALD, PCDHB15, PCDHB16, and PCDHB6) are not the same. In particular, the supplementary data of Doi et al. provides identifying information for the CpGs which indicates that they are different from the CpGs disclosed herein.

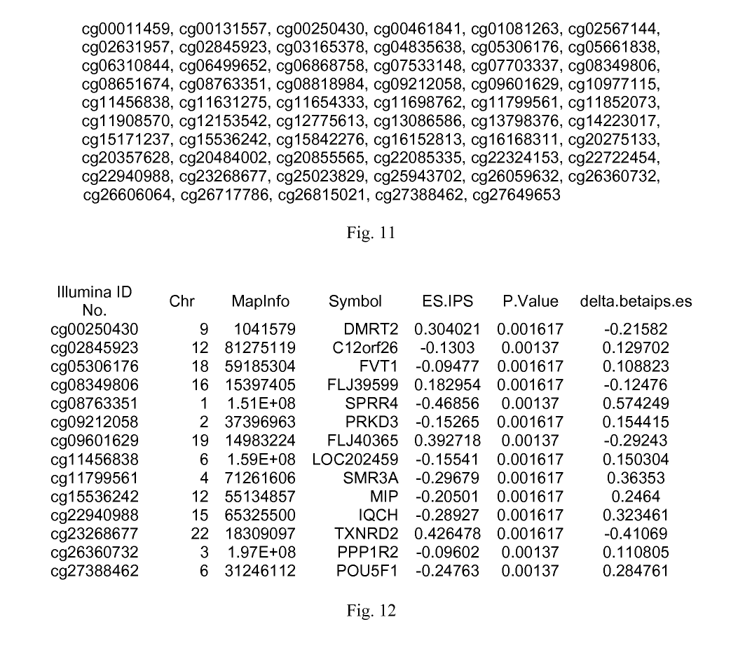

FIG. 11 shows that 59 CpGs (P<0.005 in beta value difference) out of the original 91 CpG (FIG. 10) can effectively identify or distinguish iPSCs from either hESCs or parental somatic cells.

Surprisingly, it was found that the profiles from as few as 14 CpGs can also effectively identify or distinguish iPSCs from either hESCs or parental somatic cells. As shown in FIG. 12, these 14 CpGs out of the original 91 CpGs (FIG. 10) can be used to effectively distinguish three cell types based on their unique methylation signatures.

To test whether methylation signatures discovered in our experiments can be obtained and judged with other experimental approaches, we first correlate beta values from the INFINIUM HUMANMETHYLATION27 BEADCHIP with the percentage of DNA methylation in each CpG. Based on the analysis in FIG. 4, the Illumina beta value was converted as follows: beta≧0.85, 0.85>beta≧0.7, 0.7>beta≧0.4, 0.4>beta≧0.17, 0.17>beta≧0.1, or beta≦0.1 will correspond to percentage of DNA methylation as 100%, 75%, 50%, 20%, 5%, and 0%, respectively. Then a model for cell type classification using methylation level data measured by either the beta values through Illumina microarray or by conventional methods such as bisulfite sequencing or quantitative methylation-specific PCR was built.

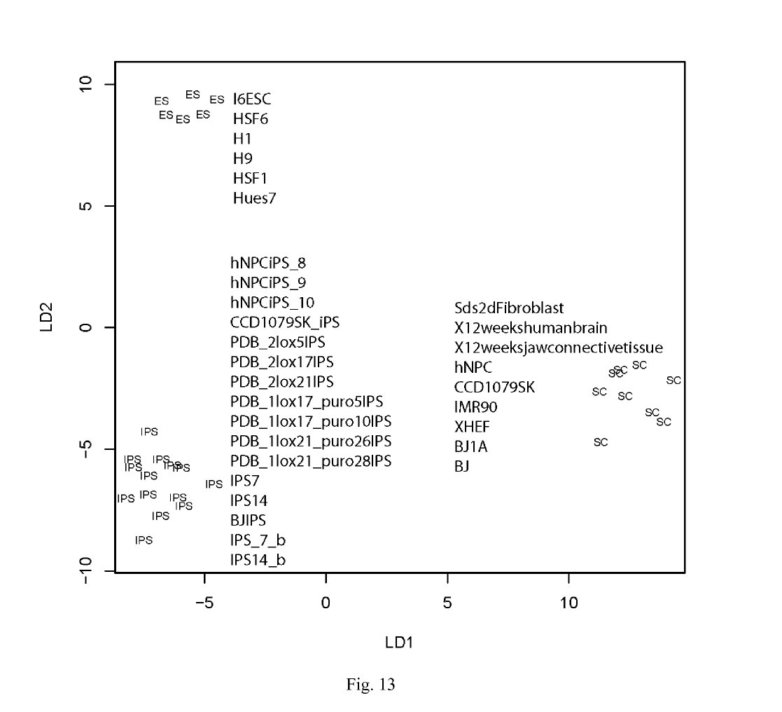

Based on results in FIG. 13, a total 14 CpGs were used for analysis, including cg27388462, cg02845923, cg08763351, cg09601629, cg26360732, cg05306176, cg00250430, cg11799561, cg09212058, cg11456838, cg08349806, cg22940988, cg15536242, and cg23268677.

Linear discriminant analysis is a method used in statistics and machine learning to find a linear combination of variables (CpGs in the instant case) which separate two or more classes of objects. The resulting combination can be used for object classification (cell types in the instant case) and prediction of new coming observation. An observation is classified into a group if the squared distance of observation to the group center is the minimum and thus the observation has the largest linear discriminant function.

After training the model using the data of FIG. 12, the squared distance formula for each group (i.e ESCs, iPSCs, SCs) was obtained. This formula was used to classify our samples with 100% correction. See FIG. 13. To compensate for an optimistic apparent error rate, the cross-validation was run by omitting each observation one at a time, recalculating the squared distance formula using the remaining data, and then classifying the omitted observation. The cross-validation was also with 100% correction in classification. This indicates that the formula below can be used to classify the three cell types without an error, i.e. 100% accuracy. The classification formula below can correctly classify any sample into a correct cell type given a sample with methylation level data of the above 14 CpGs in percentage format.

| | ES | IPS | SC |
|---|---|---|---|
| constant | −352.08 | −592.69 | −232.88 |
| cg27388462 | 0.36 | 1.99 | 2.3 |
| cg02845923 | −0.22 | −0.29 | 0.31 |
| cg08763351 | 0.07 | 0.67 | 0.72 |
| cg09601629 | −4.01 | −4.69 | −1.59 |
| cg26360732 | −0.03 | 0.18 | −0.12 |
| cg05306176 | 1.06 | 2.12 | 1.84 |
| cg00250430 | 0.43 | −0.2 | −0.22 |
| cg11799561 | −0.91 | −1.21 | −0.85 |
| cg09212058 | 2.43 | 4.25 | 2.85 |
| cg11456838 | 2.26 | 2.19 | 0.52 |
| cg08349806 | 7.21 | 8.2 | 2.79 |
| cg22940988 | −0.63 | −0.56 | 0 |
| cg15536242 | 2.74 | 3.42 | 1.53 |
| cg23268677 | 0.98 | 0.59 | −0.04 |

It should be noted that the constant values for a particular group of cells (e.g. ESC, IPS and SC) is obtained for a predetermined set of CpGs. Then the methylation levels of the same predetermined set of CpGs are determined for an unknown cell to be characterized. In other words, constant values determined for one set of CpGs can not be applied to a different set of CpGs.

### Experimental Procedures

Derivation and cultures of human iPSCs and hESCs: Human iPSCs were generated from newborn foreskin fibroblasts (CCD-1097SK and BJ1, ATCC, Rockville, Md.), fetal lung fibroblasts (IMR90, ATCC, Rockville, Md.), and neural precursor cells (hNPCs, Shen et al. (2006) Human Molec. Genetics 15:2623-2635, which is herein incorporated by reference in its entirety) with either OSKM or the combination of OCT4, NANOG, Klf4, and LIN-28 (ONKL, Yu et al. (2007) Human Molec. Genetics 15(17):2623-2635, which is herein incorporated by reference in its entirety). The hNPCs were found to be more amendable to reprogramming because the efficiency of converting hNPCs into iPSCs with the same OSKM retroviruses were two-fold that of mouse embryonic fibroblasts and eighty times that of human foreskin fibroblasts. All the newly generated human iPSCs exhibited characteristic features of hESCs, including the expression of pluripotency markers OCT4, NANOG, SSEA4 and alkaline phosphatase (data not shown); and the ability to differentiate into the derivatives of three germ layers in in vivo teratoma formation and in vitro differentiation experiments (data not shown).

The production of human iPSCs follows up the protocol described by Takahashi et al. (2007) and Yu et al. (2007) using retroviruses expression OCT4, SOX2, KLF4, and c-MYC or OCT4, NANOG, KLF4 and LIN-28. See Takahashi et al. (2007) Cell 131:861-872; and Yu et al. (2007) Science 318: 1917-1920, which are herein incorporated by reference in their entirety. hESC cells were maintained in DME supplemented with 20% KSR, nonessential amino acids (Invitrogen, Carlsbad, Calif.), L-Glutamine (Mediatech, Manassas, Va.), Penn/Strep, 2-mercaptoethanol with a feeder layer of MEFs as previously described. See Shen et al. (2006) Human Molec. Genetics 15:2623-2635, which is herein incorporated by reference in its entirety. For both gene expression and methylation analysis studies, the mESCs were passaged onto feeder free gelatin coated plates twice before harvesting RNA and DNA. RNA was isolated using Trizol (Invitrogen, Carlsbad, Calif.) while DNA was isolated using PureLink™ genomic DNA purification kit (Invitrogen, Carlsbad, Calif.).

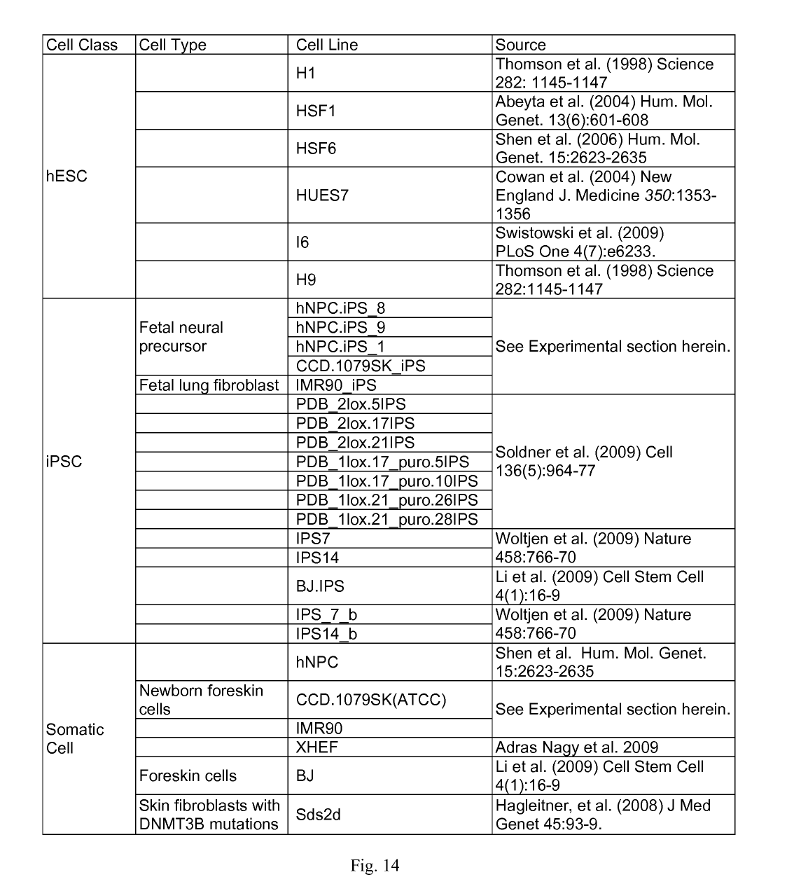

The cell lines as exemplified herein are set forth in FIG. 14.

DNA methylation profiling with Illumina Infinium assays: HUMANMETHYLATION27 BEADCHIP arrays from Illumina, Inc. (San Diego, Calif.) were used to interrogate 26,837 highly informative CpGs over 14,152 genes. Human DNA sequence was based on NCBI CCDS database (Genome Build 36) as described by the manufacturer. The experimental procedures of bisulfite conversion of genomic DNAs, hybridization of HUMANMETHYLATION27 BEADCHIP arrays, and extraction of raw hybridization signals followed manufacturer's instruction. Data analysis was performed with the BEADSTUDIO software from Illumina, Inc. (San Diego, Calif.).

Clustering analysis of methylation data: Cluster analysis of methylation data was performed by using METHYLATION MODULE v1.0 in BEADSTUDIO (Illumina, Inc., San Diego, Calif.) according to the manufacturer's manual. Briefly, average signals of built-in negative control were used as the background value to normalize the methylation signals. Outliers are removed by using the median absolute deviation method. Methylation level of individual loci in individual samples and sample groups was presented as beta value, which is estimated by calculating the ratio of intensities between methylated and unmethylated alleles. Differential methylation analysis algorithms and error models inherent in BEADSTUDIO were used to compare ES group samples and iPSCs (reference group). Samples with significant differences of delta-beta value>|0.3|, |0.4|, |0.5| were selected and subjected to the clustering analysis using cluster methods built in the BEADSTUDIO software (Illumina, Inc., San Diego, Calif.).

Bisulfite Conversion and Sequencing: Bisulfite conversion was performed as previously described. See Shen et al. (2006) Human Molec. Genetics 15:2623-2635; and Fouse et al. (2008) Cell Stem Cell 2:160-169, which are herein incorporated by reference in their entirety. Briefly, genomic DNA was digested with BglII overnight. Digested DNAs were then incubated with a sodium bisulfite solution for 16 hours. Bisulfite treated DNA was then desalted and precipitated. For each PCR, 1/10 of precipitated DNA was used. For PCR, nested primers were used to generate amplified PCR products. PCR products were gel purified and used for either Topo Cloning (Invitrogen, Carlsbad, Calif.).

Whole-genome gene expression analysis: Gene expression microarrays were done with Whole-Genome expression HUMANHT-12 BEADCHIP microarrays (HUMANREF-8 v3.0 EXPRESSION BEADCHIP, Illumina, Inc., San Diego, Calif.) using the suggested protocol. BEADSTUDIO Software was used for data analysis.

Quantitative Real-time PCR: RNA was DNase I treated (Invitrogen, Carlsbad, Calif.) and then quantified again. cDNA conversion was done using the ISCRIPT kit (BioRad, Hercules, Calif.). Quantitative PCR was done on a MYIQ Thermocycler (Biorad, Hercules, Calif.) using the SYBR GREEN SUPERMIX (BioRad, Hercules, Calif.). Relative expression levels were normalized to 18s amplicons.

Classifying Cells as ESCs, iPSCs, and Somatic Cells (SC): based on quantitative measure of levels of methylation using the methylation profile of a set of CpGs was conducted as follows. To quantitatively classify three types of cells (embryonic stem cells (ESC), induced pluripotent stem (iPSC), and somatic cells (SC)) based on a unique methylation signature, a linear discriminant model from selected beta values that are distinct for given cell types was developed. To generalize this cell type classification model as a function of methylation levels, the beta value, beta≧0.85, 0.85>beta≧0.7, 0.7>beta≧0.4, 0.4>beta≧0.17, 0.17>beta≧0.1, or beta<0.1 to percentage of DNA methylation was expressed as a percentage of methylation, 100, 75, 50, 20, 5, and 0, respectively. See Matrix Table below.

This conversion was based on the linear regression of beta values against true methylation levels measured by bisulfite-treated DNA sequencing (FIG. 4). The advantage of using the percentage of DNA methylation is that this model may be universally useful for classifying cells purely based on DNA methylation levels as measured by either Illumina microarray (such as INFINIUM HUMANMETHYLATION27 BEADCHIP) assays, or bisulfite sequencing, or any other quantitative methylation assay including pryos-sequencing or mass-spectrometry.

As exemplified herein, a total of 14 CpGs, including cg27388462, cg02845923, cg08763351, cg09601629, cg26360732, cg05306176, cg00250430, cg11799561, cg09212058, cg11456838, cg08349806, cg22940988, cg15536242, and cg23268677 that are selected from the 59 CpGs of FIG. 11.

Linear discriminant analysis is a method used in statistics and machine learning to find a linear combination of variables (CpGs, in this case) which separate two or more classes of objects. The resulting combination can be used for both object classification (cell types, in is case) and prediction of new observations. An observation is classified into a group by finding the minimum of the squared distance of the observation to a group's center, and thus also having the largest linear discriminant function. The following linear discriminant function was used to obtain a constant (k) value for each class of cells (i.e ESC, iPSC, SC) for the given set of CpGs:

$${δ}_{k} ( x ) = {x}^{T} {Σ}^{- 1} {μ}_{k} - \frac{1}{2} {μ}_{k}^{T} {Σ}^{- 1} {μ}_{k} + log {π}_{k}$$

\hat{π}ₖ=Nₖ/N, where Nₖ is the number of class-k observations;

\hat{μ}ₖ=Σ_{qi}₌ₖxᵢ/Nₖ;

\hat{Σ}=Σₖ₌₁^{K}Σ_{gi}₌ₖ(xᵢ−\hat{μ}ₖ)(xᵢ−\hat{μ}ₖ)^{T}/(N−K).

x is the observation.

After training the model using the data herein for the given set of CpGs, the squared distance formula, including constants and regression coefficients, was calculated for each group (i.e ESC, iPSC, SC) using a matrix table as set forth below:

| Matrix Table | cg27388462 | cg02845923 | cg08763351 | cg09601629 | cg26360732 | cg05306176 | cg00250430 | cg11799561 |
|---|---|---|---|---|---|---|---|---|
| I6ESC | 20 | 50 | 50 | 100 | 5 | 5 | 50 | 50 |
| Sds2dFibroblast | 50 | 75 | 50 | 5 | 0 | 20 | 20 | 20 |
| X12weekshumanbrain | 75 | 75 | 75 | 5 | 0 | 5 | 5 | 50 |
| X12weeksjawconnectivetissue | 20 | 75 | 75 | 5 | 0 | 20 | 5 | 50 |
| hNPCiPS_8 | 50 | 100 | 75 | 5 | 5 | 5 | 0 | 75 |
| hNPCiPS_9 | 50 | 100 | 75 | 5 | 5 | 5 | 5 | 75 |
| hNPCiPS_10 | 50 | 100 | 75 | 5 | 20 | 5 | 0 | 75 |
| hNPC | 50 | 100 | 75 | 5 | 5 | 5 | 5 | 75 |
| CCD1079SK_iPS | 50 | 100 | 75 | 5 | 5 | 5 | 0 | 75 |
| CCD1079SK | 50 | 75 | 20 | 0 | 0 | 5 | 20 | 50 |
| HSF6 | 20 | 75 | 0 | 20 | 0 | 5 | 0 | 20 |
| H1 | 20 | 50 | 50 | 5 | 0 | 5 | 50 | 20 |
| H9 | 20 | 75 | 20 | 50 | 0 | 5 | 50 | 50 |
| HSF1 | 20 | 75 | 20 | 75 | 0 | 5 | 0 | 50 |
| Hues7 | 20 | 75 | 5 | 5 | 5 | 5 | 20 | 50 |
| IMR90 | 75 | 100 | 20 | 5 | 0 | 5 | 0 | 50 |
| PDB_2lox5IPS | 50 | 100 | 75 | 5 | 5 | 20 | 0 | 75 |
| PDB_2lox17IPS | 50 | 75 | 75 | 5 | 5 | 20 | 0 | 75 |
| PDB_2lox21IPS | 50 | 75 | 75 | 5 | 5 | 20 | 0 | 75 |
| PDB_1lox17_puro5IPS | 50 | 75 | 75 | 5 | 5 | 20 | 0 | 75 |
| PDB_1lox17_puro10IPS | 50 | 100 | 75 | 5 | 5 | 20 | 0 | 75 |
| PDB_1lox21_puro26IPS | 50 | 75 | 75 | 5 | 5 | 20 | 0 | 75 |
| PDB_1lox21_puro28IPS | 50 | 75 | 75 | 5 | 5 | 20 | 0 | 75 |
| XHEF | 50 | 75 | 50 | 5 | 0 | 20 | 5 | 50 |
| IPS7 | 50 | 75 | 75 | 5 | 5 | 5 | 0 | 75 |
| IPS14 | 50 | 75 | 75 | 5 | 20 | 5 | 0 | 50 |
| BJ1A | 50 | 75 | 20 | 5 | 5 | 5 | 20 | 50 |
| BJIPS | 50 | 75 | 75 | 5 | 5 | 20 | 0 | 50 |
| IPS_7_b | 50 | 75 | 75 | 5 | 5 | 5 | 0 | 75 |
| IPS14_b | 50 | 75 | 75 | 5 | 20 | 20 | 0 | 50 |
| BJ | 50 | 100 | 20 | 5 | 5 | 5 | 20 | 50 |

| Matrix Table | cg09212058 | cg11456838 | cg08349806 | cg22940988 | cg15536242 | cg23268677 |
|---|---|---|---|---|---|---|
| I6ESC | 50 | 50 | 100 | 50 | 50 | 100 |
| Sds2dFibroblast | 50 | 75 | 0 | 50 | 50 | 5 |
| X12weekshumanbrain | 50 | 75 | 5 | 50 | 50 | 0 |
| X12weeksjawconnectivetissue | 75 | 50 | 5 | 50 | 50 | 5 |
| hNPCiPS_8 | 75 | 75 | 50 | 75 | 75 | 50 |
| hNPCiPS_9 | 100 | 75 | 50 | 75 | 75 | 50 |
| hNPCiPS_10 | 75 | 75 | 50 | 75 | 75 | 50 |
| hNPC | 75 | 75 | 5 | 100 | 75 | 5 |
| CCD1079SK_iPS | 75 | 75 | 50 | 75 | 75 | 50 |
| CCD1079SK | 75 | 75 | 5 | 100 | 50 | 5 |
| HSF6 | 50 | 50 | 50 | 50 | 50 | 100 |
| H1 | 50 | 50 | 50 | 20 | 20 | 75 |
| H9 | 75 | 75 | 50 | 50 | 50 | 100 |
| HSF1 | 50 | 75 | 75 | 50 | 50 | 100 |
| Hues7 | 50 | 50 | 50 | 50 | 50 | 75 |
| IMR90 | 50 | 50 | 20 | 50 | 50 | 5 |
| PDB_2lox5IPS | 75 | 75 | 50 | 75 | 75 | 20 |
| PDB_2lox17IPS | 75 | 100 | 50 | 75 | 75 | 20 |
| PDB_2lox21IPS | 75 | 75 | 50 | 75 | 75 | 50 |
| PDB_1lox17_puro5IPS | 75 | 75 | 50 | 75 | 75 | 20 |
| PDB_1lox17_puro10IPS | 75 | 100 | 50 | 100 | 75 | 20 |
| PDB_1lox21_puro26IPS | 75 | 75 | 50 | 100 | 75 | 20 |
| PDB_1lox21_puro28IPS | 75 | 75 | 50 | 75 | 75 | 50 |
| XHEF | 50 | 50 | 5 | 75 | 75 | 5 |
| IPS7 | 75 | 75 | 50 | 75 | 75 | 20 |
| IPS14 | 75 | 75 | 50 | 75 | 75 | 20 |
| BJ1A | 75 | 50 | 5 | 100 | 50 | 5 |
| BJIPS | 75 | 75 | 50 | 75 | 75 | 50 |
| IPS_7_b | 75 | 75 | 50 | 75 | 75 | 20 |
| IPS14_b | 75 | 75 | 50 | 75 | 75 | 20 |
| BJ | 75 | 50 | 5 | 100 | 75 | 5 |

In the following Table, the values indicated for the CpGs are coefficients which are obtained similarly using simple linear regression y=ax+b, b-constant, and a-coefficient. The constants and coefficients shown in the Table below were calculated by the known dataset based on the linear discriminant analysis function 1 as provided above.

| | ESC | iPSC | SC |
|---|---|---|---|
| constant | −352.08 | −592.69 | −232.88 |
| cg27388462 | 0.36 | 1.99 | 2.3 |
| cg02845923 | −0.22 | −0.29 | 0.31 |
| cg08763351 | 0.07 | 0.67 | 0.72 |
| cg09601629 | −4.01 | −4.69 | −1.59 |
| cg26360732 | −0.03 | 0.18 | −0.12 |
| cg05306176 | 1.06 | 2.12 | 1.84 |
| cg00250430 | 0.43 | −0.2 | −0.22 |
| cg11799561 | −0.91 | −1.21 | −0.85 |
| cg09212058 | 2.43 | 4.25 | 2.85 |
| cg11456838 | 2.26 | 2.19 | 0.52 |
| cg08349806 | 7.21 | 8.2 | 2.79 |
| cg22940988 | −0.63 | −0.56 | 0 |
| cg15536242 | 2.74 | 3.42 | 1.53 |
| cg23268677 | 0.98 | 0.59 | −0.04 |

Then for an unknown cell, the amounts of methylation for each of the CpGs are determined and used to solve the following equations:

ESC=−352.08+0.36*cg27388462 (i.e. % of methylation of cg27388462 e.g. 75% would be 75)−0.22*cg02845923+0.07*cg08763351−4.01*cg09601629−0.03*cg26360732+1.06*cg05306176+0.43*cg00250430−0.91*cg11799561+2.43*cg09212058+2.26*cg11456838+7.21*cg08349806−0.63*cg22940988+2.74*cg15536242+0.98*cg23268677.

iPSC=−592.69+1.99*cg27388462 . . . +0.59*cg23268677.

SC=−232.88+2.3*cg27388462 . . . −0.04*cg23268677.

The three parallel calculations yield three values. The largest value indicates the class to which the unknown cell belongs. For example, if the iPSC equation yields the largest number, the unknown cell is then characterized as an iPSC.

In addition, linear discriminant component 1 and 2 (LD1 and LD2, respectively) was also derived from above model. LD1 and LD2 are derived by finding the best possible angle to view a multi-dimensional space (31 dimensions from 31 cell lines in this case). Best possible angle is determined by finding the maximum variance between all data points (i.e. contains most information). After finding the best angle, the data is “projected” to 2 or 3-dimensional space (2 dimension in this case), which is expressed as LD1 and LD2. To determine LD1 and LD2 of an unknown cell, linear discriminant analysis is run alongside the known classifications (31 known cell lines) to determine how the unknown cell fits with the known dataset. With the constants and coefficients (i.e. the best angle) are in hand, one multiplies the constants and coefficients with the unknown cell's methylation level data to determine which space (quadrant) the unknown cell belongs as described above, with LD1 and LD2 coordinates. This formula was used to classify our samples with 100% correction. See FIG. 13. To compensate for an optimistic apparent error rate, cross-validation by systematically omitting each observation, recalculating the squared distance formula using the remaining data, and re-classifying the omitted observation was run. The cross-validation was also performed with 100% correction in classification. This indicates the formula above can be used to classify the three cell types with very little error. In summary, this classification formula can correctly classify any sample into a correct cell type given a sample with methylation level data of the above 14 CpG sites in percentage format.

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

1. A method of characterizing a cell as being of a particular cell type from a predetermined group of cell types comprising cell type A and cell type B which comprises
conducting the method of claim 4,
obtaining cell type A methylation profiles of a set of known cells of cell type A for a set of CpGs;
obtaining cell type B methylation profiles of a set of known cells of cell type B for the set of CpGs;
obtaining an amount of methylation for each CpG of the set of CpGs for the cell;
using linear discriminant analysis to obtain a cell type A constant value and a cell type B constant value from the cell type A methylation profiles and the cell type B methylation profiles, respectively;
using linear discriminant analysis to obtain a first set of coefficients which correspond to cell type A for the set of CpGs;
using linear discriminant analysis to obtain a second set of coefficients which correspond to cell type B for the set of CpGs;
calculating a first value by multiplying each of the amounts of methylation with the corresponding coefficients of the first set of coefficients to obtain a first set of multiplied values, summing the first set of multiplied values, and adding the cell type A constant value;
calculating a second value by multiplying each of the amounts of methylation with the corresponding coefficients of the second set of coefficients to obtain a second set of multiplied values, summing the second set of multiplied values, and adding the cell type B constant value;
designating the cell as being of cell type A where the first value is greater than the second value or designating the cell as being of cell type B where the second value is greater than the first value.

2. The method of claim 1, wherein the predetermined cell types includes additional cell types and the methylation amounts of the additional cell types are similarly determined and compared with the methylation amount of the cell.

3. A method of characterizing a cell as an embryonic stem cell, an induced pluripotent stem cell or a somatic cell which comprises
conducting the method according to claim 4,
obtaining embryonic stem cell methylation profiles from a set of known embryonic stem cell for a set of CpGs;
obtaining induced pluripotent stem cell methylation profiles from a set of known induced pluripotent stem cell for the set of CpGs;
obtaining somatic cell methylation profiles of a set of known somatic cell for the set of CpGs;
obtaining an amount of methylation for each CpG of the set of CpGs for the cell;
using linear discriminant analysis to obtain an ESC constant value, an iPSC constant value, and an SC constant value from the embryonic stem cell methylation profiles, induced pluripotent stem cell methylation profiles, and somatic cell methylation profiles, respectively;
using linear discriminant analysis to obtain a first set of coefficients which correspond to the embryonic stem cell methylation profiles for the set of CpGs, a second set of coefficients which correspond to the induced pluripotent stem cell methylation profiles for the set of CpGs, and a third set of coefficients which correspond to the somatic cell methylation profiles for the set of CpGs;
calculating a first value by multiplying each of the amounts of methylation with the corresponding coefficients of the first set of coefficients to obtain a first set of multiplied values, summing the first set of multiplied values, and adding the ESC constant value;
calculating a second value by multiplying each of the amounts of methylation with the corresponding coefficients of the second set of coefficients to obtain a second set of multiplied values, summing the second set of multiplied values, and adding the iPSC constant value;
calculating a third value by multiplying each of the amounts of methylation with the corresponding coefficients of the third set of coefficients to obtain a third set of multiplied values, summing the third set of multiplied values, and adding the SC constant value;
designating the cell as being an embryonic stem cell where the first value greater than the second and third values, designating the cell as being an induced pluripotent stem cell where the second value is greater than the first and third values, or designating the cell as being a somatic cell where the third value is greater than the first and second values.

4. A method of characterizing a cell which comprises
determining a methylation state of at least one CpG in a region of a nucleotide molecule of the cell,
comparing the methylation state with that of a corresponding CpG of a comparison cell of a known cell type, a known cell line, or a known cell strain, and
distinguishing, identifying or designating the cell type, the cell line or the cell strain of the cell based on whether the methylation state is the same or different from that of the corresponding CpG.

5. The method of claim 4, wherein the cell is distinguished from the comparison cell where the methylation state is different from that of the corresponding CpG.

6. The method of claim 4, wherein the cell type, the cell line or the cell strain of the cell is identified as being the known cell type or the known cell line where the methylation state is the same or substantially similar to that of the corresponding CpG.

7. The method of claim 4, wherein the cell type, the cell line or the cell strain of the cell of the cell is designated as being that of the comparison cell where the methylation state is the same or substantially similar to that of the corresponding CpG.

8. The method of claim 4, wherein the CpG is in a CpG island, outside of a CpG island, or a promoter.

9. The method of claim 4, wherein the methylation states of more than one CpG are determined and compared to the methylation states of the corresponding CpGs of the comparison cell.

10. The method according to claim 4, wherein the CpG is selected from the group consisting the CpGs of FIG. 6.

11. The method according to claim 4, wherein the cell type is selected from the group consisting human embryonic stem cells (hESCs), a neural precursor cell, human induced pluripotent stem cells, fetal lung fibroblasts, fetal brain tissues, foreskin fibroblasts, fetal lung fibroblasts, differentiated neural precursor cells from hESCs, neural precursor cells differentiated from human iPSCs, retinal pigment epithelial cells from hESCs, and neurons derived from hESCs.

12. The method according to claim 4, wherein the cell type or cell line is selected from the group consisting of H1, HSF1, HSF6, HUES7, H9, I6, hNPC.iPS_{—}8, hNPC.iPS_{—}9, hNPC.iPS_{—}1, CCD.1079SK_iPS, PDB_{—}2lox.5IPS, PDB_{—}2lox.17IPS, PDB_{—}2lox.21IPS, PDB_{—}1lox.17_puro.5IPS, PDB_{—}1lox.17_puro.10IPS, PDB_{—}1lox.21_puro.26IPS, PDB_{—}1lox.21_puro.28IPS, IPS7, IPS14, BJ.IPS, IPS_{—}7_b, IPS14_b, hNPC, CCD.1079SK, IMR.9, XHEF, BJ, and sds2d.

13. The method according to claim 4, wherein the methylation states of at least 91 CpGs are determined and compared with the corresponding CpGs.

14. A method of characterizing a cell as being an induced pluripotent cell or an embryonic stem cell which comprises
determining the methylation states of at least 14 CpG selected from Group A consisting of cg11852073, cg26606064, cg06868758, cg08349806, cg00250430, cg05661838, cg20855565, cg09601629, cg12153542, cg20357628, cg23268677, cg15842276, cg15747595, and cg07533148
and/or Group B consisting of cg08763351, cg13798376, cg00461841, cg11328541, cg11799561, cg26059632, cg22940988, cg21021629, cg03165378, cg25539131, cg08818984, cg27388462, cg05950276, cg01804844, cg25423111, cg11378044, cg18118795, cg06509940, cg27649653, cg10977115, cg12775613, cg15536242, cg22324153, cg26866325, cg20484002, cg21784940, cg02245418, cg13086586, cg16168311, cg14223017, cg18509239, cg11908570, cg12368241, cg26815021, cg08028004, cg23504707, cg11456838, cg09212058, cg07703337, cg25023829, cg10742225, cg01796228, cg02845923, cg16152813, cg15171237, cg25943702, cg00011459, cg16546489, cg11631275, cg20275133, cg00131557, cg04835638, cg25156443, cg07105440, cg26360732, cg05306176, cg06310844, cg24562819, cg15864184, cg15781794, cg01081263, cg26014197, cg11368509, cg08651674, cg00376639, cg22722454, cg26717786, cg06499652, cg11698762, cg22085335, cg23943801, cg02631957, cg21260850, cg24471268, cg11654333, cg11594131, and cg02567144, and
identifying or designating the cell as being an induced pluripotent cell where the CpG is selected from Group A and is methylated or where the CpG is selected from Group B and is unmethylated, or identifying or designating the cell as being an embryonic stem cell where the CpG is selected from Group A and is unmethylated or where the CpG is selected from Group B and is methylated.

15. A method of determining whether a first cell is the same or different from a second cell which comprises
determining a first methylation profile of the first cell for a plurality of CpGs consisting of at least 11 to 273 CpGs selected from the group consisting of the CpGs of FIG. 6,
determining a second methylation profile of the second cell for the plurality of CpGs,
comparing the first methylation profile with the second methylation profile, and
designating the first cell and the second cell to be the same where the first methylation profile and the second methylation profile are the same or designating the first cell and the second cell to be different where the first methylation profile and the second methylation profile are different.
